(19) United States
(12) Patent Application Publication
(10) Pub. No.: US 2014/0377789 A1
(43) Pub. Date: Dec. 25, 2014

(54) ASSAY AND METHOD FOR DETERMINING INSULIN-RESISTANCE

(71) Applicant: ALERE SWITZERLAND GMBH, Zug (CH)

(72) Inventor: Piet Moerman, Deurle (BE)

(21) Appl. No.: 14/373,777

(22) PCT Filed: Feb. 8, 2013
(86) PCT No.: PCT/EP2013/052512

§ 371 Date: Jul. 22, 2014

(30) Foreign Application Priority Data
Feb. 10, 2012 (EP) PCT/EP2012/052289
Jan. 24, 2013 (GB) 1301330.5

(51) Int. Cl. C12Q 1/54; G01N 27/327; G01N 33/66; G01N 27/416

(52) U.S. Cl. CPC C12Q 1/54; G01N 27/416; G01N 27/3271; G01N 33/66
USPC 435/14; 204/403.01; 204/403.14; 205/792; 435/288.7

(57) ABSTRACT

The present invention provides for a home test or a point of care test device that can both detect blood glucose and insulin levels and methods using said device. The device and methods can be used to aid diabetic patients and medical practitioners to fine tune insulin administration, and to monitor disease progression or treatment.

### FIELD OF THE INVENTION

The present invention is situated in the field of medical diagnostics, more in particular in the field of diagnosis of insulin need or insulin resistance, based on the simultaneous detection of insulin and glucose levels in a whole blood sample of the subject.

### BACKGROUND OF THE INVENTION

In 2005-2008, based on fasting glucose or hemoglobin A1c levels, 35% of U.S. adults aged 20 years or older had pre-diabetes (50% of adults aged 65 years or older). Applying this percentage to the entire U.S. population in 2010 yields an estimated 79 million American adults aged 20 years or older with prediabetes. After adjusting for population age and sex differences, average medical expenditures among people with diagnosed diabetes were 2.3 times higher than what expenditures would be in the absence of diabetes. Being able to prevent the development of actual diabetes in said population is hence a huge challenge.

In addition, in type-1 diabetes mellitus (T1DM) patients, knowing one's insulin sensitivity when and where needed is today still largely based on inaccurate assumptions. This is due to the cumbersome and expensive insulin tests, which have to be carried out in a lab. The insulin quantity for the bolus injection just before the meal is calculated using the actual glucose level measured using a home glucose test, in combination with the insulin sensitivity or resistance that was estimated some time ago by deduction. This insulin sensitivity factor is however not at all reflecting the actual insulin sensitivity at the time of the bolus injection or the time of measuring the blood glucose. The resulting output of the calculated insulin bolus quantity (or dosage) is hence often inaccurate and may lead to non-healthy or dangerous glucose and/or insulin levels.

Also for type-2 diabetes mellitus (T2DM) patients, the actual real insulin-sensitivity cannot be calculated by the patient himself, since he is currently not able to obtain real-time data on the blood-insulin level.

The combined insulin and glucose level is hence essential information, not only for patients with type 1 and type 2 diabetes mellitus, but also for patients with Metabolic Syndrome and excess body weight, since these pathologies precede diabetes and are often linked to insulin resistance.

Insulin resistance is the 1/insulin sensitivity and is in fact the reciprocal of insulin sensitivity.

Currently an insulin test has to be shipped to a central clinical lab since there is no point of care test (POC test) on the market. The POC and self-monitoring markets are not developed. Only recently, technologies which may make the detection and quantification of pmol/L quantities of analyte in one drop-size sample possible in a user friendly POC-layout, have emerged.

Although many blood glucose tests and sensors are currently available for home use, to our knowledge no blood insulin test for home use or as a point of care device has been reported, nor has this been combined with a glucose test in a single device. The present invention intends to overcome this need.

### SUMMARY OF THE INVENTION

The present invention provides products and methods that combine the testing of both glucose and insulin levels in a blood sample of a subject and immediately calculate the insulin resistance (IR), insulin sensitivity (IS) or beta-cell function from it. The product is to be seen as a home self test or as a point of care device for the medical practitioner. Making insulin resistance information available, when and where needed, allows for a better preservation of β-cell function (the insulin producing cells) in the overweight patient and thus contributes to the prevention of type 2 diabetes mellitus (T2DM).

The invention thus provides a device for detecting both the glucose and insulin level in a whole blood sample of a subject comprising:

a) a sample receiving part;

b) an analyte reaction zone comprising

b1) a first sensor for detecting the blood glucose level in said sample,

b2) a second sensor for detecting the blood insulin level in said sample,

c) a controlling device that can control the operation of the device and analyse the data obtained from the biosensor systems.

d) a user interface, displaying the data to the user.

In a preferred embodiment, said second sensor b2) comprises two separate sensors, one for detecting endogenous insulin or its cleaved C-peptide fragment, and one for detecting exogenous insulin. The exogenous insulin can be fast-acting or slow acting. Preferably fast-acting and slow acting (or long acting or basal insulin) can be measured separately by the device.

In another preferred embodiment, said second sensor b2) comprises two separate sensors, one for detecting fast-acting insulin and one for detecting slow acting insulin (long acting or basal insulin).

In a preferred embodiment, the analyte reaction zone b) comprises at least two tracts, one for detecting blood glucose, and one for detecting blood insulin, wherein the latter can also comprise different tracts, for detecting different types of insulin (endogenous, short-acting, and/or long-acting).

In a preferred embodiment of the device according to the invention, the glucose and insulin are measured using a single sensor system, or using two separate sensor systems to detect each analyte separately.

In a preferred embodiment, the device according to the invention is a home test device or a point of care device.

In a preferred embodiment of the device according to the invention, said insulin sensor is specifically detecting long-acting insulin, short-acting insulin, or both, or is specifically detecting C-peptide cleaved from endogenously produced insulin.

Preferably, said first sensor is an electrochemical or optical sensor, and/or said second sensor is an electrochemical or optical sensor. Preferably, both sensors are electrochemical sensors. Alternatively, both sensors are optical sensors. Combined optical/electrochemical sensors are also envisaged by the invention.

In a preferred embodiment of the device according to the invention, the detection of both the glucose and insulin level is done in a sample volume of less than 1 ml, preferably less than 0.5 ml, more preferably in less than 100 μl, most preferably in less than 10 μl, or in about 5 μl of whole blood.

In a preferred embodiment, the device according to the invention has a sensitivity of 100 pmol/l, preferably of 50 pmol/l, more preferably of 20 pmol/l or less for insulin.

In a preferred embodiment, the device according to the invention has a sensitivity of 20 mmol/L or less for glucose.

In a preferred embodiment of the device according to the invention, the controller device calculates the insulin-resistance, insulin sensitivity or beta-cell function of the subject based on the signals obtained from sensors b1) and b2). In a preferred embodiment, said calculation is done using the HOMA1-IR, HOMA2-IR, or HOMA B %, Gutt index, Avignon Index, Stumvoll Index, Matsuda Index, or HOMA B %, or the Oral Disposition Index formula to determine insulin resistance and beta-cell function in a subject.

In a preferred embodiment of the device according to the invention, said first sensor for detecting blood glucose is a glucose-oxidase or dehydrogenase based electrochemical or colorimetric system.

In a preferred embodiment of the device according to the invention, said second sensor for detecting insulin is an electrochemical sensor, measuring a change in charge or current due to enzymatic reaction with a substrate upon binding of insulin. Examples of such sensors are e.g. selected from the group comprising: electrochemical immunoassays, enzyme-activation electrochemical detection systems, enzyme-linked immunomagnetic electrochemical assays, enzyme-activation immunomagnetic electrochemical assays, and piezo-electrical or di-electrical immunoassays.

In a preferred embodiment, said second electrochemical sensor is an enzyme-linked immunomagnetic electrochemical assay comprising: an electron-releasing enzyme system coupled to an insulin-specific antibody and secondary insulin-specific antibodies, linked to magnetic particles. Preferably, upon contact with its substrate, an electron is formed by said enzyme and the current obtained through said enzymatic activity is measured. More preferably, the magnetic particles are used to capture away the insulin-bound enzyme complexes, and wherein a reduction of electronic current initially present is proportional to the amount of insulin present in the sample. More preferably, said electron-releasing enzyme system is glucose oxidase. More preferably, additionally an electron transfer mediator is used such as an ion of ferricyanide.

In a preferred embodiment, said electrochemical sensor comprises one or more electrodes or electrode couples, connected to a device capable of inducing and measuring a charge or current in either one of said electrodes.

In a preferred embodiment of the device according to the invention, said electrochemical sensor comprises one or more electrodes or electrode couples connected to a device capable of inducing and measuring a charge or current in either one of, or between said electrodes. Said charge/current device is connected and controlled by and reports to the controlling device or operating system.

In a preferred embodiment said electrodes are made of an electrically conductive material preferably selected from the group comprising: carbon, gold, platinum, silver, silver chloride, rhodium, iridium, ruthenium, palladium, osmium, copper, and mixtures thereof.

In a preferred embodiment of the device according to the invention, said electrodes are porous electrodes, magnetic electrodes, or carbon nanotubes.

In a preferred embodiment of the device according to the invention, the sample receiving part is comprised of a microporous membrane support, test strip or lateral flow test strip produced from a material selected from the group consisting of: an organic polymer, inorganic polymer, natural fabrics or synthetic fibers, papers and ceramics.

In a alternative embodiment of the test device of the invention said second sensor for detecting insulin is an optical sensor, measuring a change in color formation, light diffraction, light scattering, light adsorption, or light reflection, caused by specific binding of the analyte to the sensor.

Preferably, the optical or biochemical sensor used in the test device according to the invention, uses immunomagnetics to concentrate the analytes on the reaction zone and additionally comprising a means for inducing magnetism in said reaction and/or detection zone. Alternatively, said optical or biochemical sensor uses capillary forces for generating flow of the blood sample through the reaction zone and/or for eliminating non-bound complexes, additionally comprising an absorption pad or a capillary flow inducing means (e.g. the test strip itself). Optionally, a reservoir with fluid, connected to said reaction zone can be present to allow a better washing step.

In a preferred embodiment, the electrochemical sensor of the test device according to the invention comprises an enzyme reporting system selected from the group comprising: glucose oxidase, glucose dehydronase, hexokinase, lactate oxidase, cholesterol oxidase, glutamate oxidase, horseradish peroxidase, alcohol oxidase, glutamate pyruvate transaminase, and glutamate oxaloacetate transaminase, horseradish peroxidase/p-aminophenol immunoassay, alkaline phosphatase/1-naphthyl phosphate immunoassay. Optionally, a combination of an enzyme with an electron transfer mediator is used.

In a preferred embodiment, the device according to the invention additionally comprises an input means for introducing user-specific data such as time of measurement, time of last meal, time after exercise etc. into said controller, preferably comprising a keypad or a touch-screen, or any other means for feeding data to said device such as e.g. a wireless connection or a cable port. Said data could be fed from a PC, a portable computer, a smart phone or the like.

In a preferred embodiment, the device according to the invention, additionally comprises a connection with a computer, portable or mobile processing device, or a smart phone, to enable the user or medical practitioner to follow up his status, insulin need and beta-cell function. Said connection can be through a cable or wireless.

The invention further provides for the use of the device according to any one of the embodiments described herein, for determining the amount of insulin needed in a type-II diabetes mellitus patient, in an obese subject, or in a subject with metabolic syndrome

The invention further provides for the use of the device according to any one of the embodiments described herein, for determining the amount of insulin needed in a type-I diabetes mellitus patient

The invention further provides for the use of the device according to any one of the embodiments described herein, for evaluating the activity of the population of insulin-producing beta cells in a subject

The invention further provides for the use of the device according to any one of the embodiments described herein, for determining or evaluating the treatment of a subject with the goal to preserve the endogenous beta cell function as long as possible.

The invention further provides for the use of the device according to any one of the embodiments described herein, for measuring real time insulin sensitivity adapted insulin-to-carb ratio.

The invention further provides for the use of the device according to any one of the embodiments described herein, for measuring real time adapted insulin sensitivity glucose correction factor.

The invention further provides for the use of the device according to any one of the embodiments described herein, for measuring a real-time insulin sensitivity adapted basal rate of insulin.

The invention further provides for the use of the device according to any embodiment of the invention for determining the insulin-resistance and beta-cell function in a type 2 diabetes mellitus patient, in an obese subject, or in a subject with metabolic syndrome.

The present invention hence provides a test device and method that uses a “real-time insulin sensitivity adapted insulin-to-carb ratio” and a “real-time adapted insulin sensitivity glucose correction factor” to calculate a more appropriate bolus quantity of insulin to be administered to a subject in need thereof.

The invention further provides for a method for determining the amount of insulin needed in a type-I diabetes mellitus patient comprising the steps of:
detecting the glucose level in a blood sample of a subject,
detecting the insulin level in said sample, and
calculating the amount of insulin needed in said subject, by using the calculated Insulin sensitivity (or resistance) from the combined insulin/glucose level measured, together with the pre-meal or fasting glucose level and the quantity of carbohydrates in the next meal. In a preferred embodiment, said calculation is done using:
1. The patient's insulin to carb ratio, to calculate how much insulin is needed to absorb the carbohydrates from the next meal PLUS
2. The patient's glucose correction factor to calculate how much insulin is needed to correct the pre-meal or fasting glucose level.
3. And the patient's Insulin Resistance measured at that time.
4. Preferably also the subtraction of Insulin On Board, i.e. the amount of insulin left over in the subcutis from the previous injection

The insulin to carb ratio is the amount of insulin needed to absorb 15 grams of carbohydrates form his next meal in said subject, and the glucose correction factor is the factor of insulin needed to lower the pre-meal blood glucose level in said subject to a target range.

The invention further provides for a method for diagnosing, prognosticating, predicting or determining the disease state of a type 2 diabetes mellitus patient, an obese subject, or a subject with metabolic syndrome comprising the steps of:
determining the glucose level in a blood sample of said subject,
determining the insulin level in a blood sample of said subject,
calculating the insulin-resistance or beta-cell function based on the level of blood glucose and insulin measured, preferably using the device according to any embodiment of the invention,
determining the status of the subject, based on said insulin-resistance or beta-cell function. Typically, an increased insulin-resistance or a reduced beta-cell function is indicative of worsening of the disease state of the subject. Preferably, said insulin resistance is calculated using the HOMA1-IR or HOMA2-IR-test, and said beta-cell function is measured using the HOMA-B % test or any other function used for that purpose. See following table:

TABLE 1

| Method | Measurement | Comments |
|---|---|---|
| Matsuda index | 10 000/√ (fasting G × fasting I) | Represents both hepatic and peripheral |
| | (mean G × mean I) | tissue sensitivity to insulin |
| Gutt index | 75 000 + (G₀ − G₁₂₀) (mg/dL) × 0.19 × | Good to predict onset of type 2 diabetes |
| | BW/120 × Gmean_{(0, 120)} (mmol/L) × | |
| | Log [Imean_{(0, 120)}] (mU/L) | |
| Stumvoll index | 0.156 − 0.0000459 × I₁₂₀ (pmol/L) − | Utilizes demographic data like age, sex |
| | 0.000321 × I₀(pmol/L) − 0.00541 × | and BMI along with plasma glucose and |
| | G₁₂₀ (mmol/L) | insulin to predict insulin sensitivity |
| Avignon index | Sib = 10⁸/[I₀ (mU/L) × G₀ (mmol/L) × | Determines glucose tolerance and |
| | VD) Si2h = 10⁸/(I₁₂₀ (mU/L) × | insulin sensitivity in single test |
| | G₁₂₀ (mmol/L) × VD] | |

| | |
|---|---|
| Oral glucose | G and I concentrations from a 75 g OGTT at 0, 2, and 3 h (3 h OGTT) or at 0, |
| insulin sensitivity | 1.5, and 2 h (2 h OGTT). The formula includes six constants |
| index | |
| Log (HOMA-IR) | Evaluates insulin resistance in insulin-resistant states like glucose intolerance |
| | and mild to moderate diabetes |

Sib: Derived from fasting plasma insulin and glucose; Si2h: Derived from fasting plasma insulin and glucose and 2 h of OGTT; OGTT = Oral glucose tolerance test.

The invention further provides for a method for screening a population of subjects for being pre-diabetic or for the risk of becoming a diabetic subject, comprising the steps of:
determining the glucose level in a blood sample of said subject,
determining the insulin level in a blood sample of said subject,
calculating the insulin-resistance or beta-cell function based on the level of blood glucose and insulin measured, preferably using the device according to any embodiment of the invention, and
determining whether or not the subject is pre-diabetic or has a risk of becoming a diabetic, based on said insulin-resistance or beta-cell function.

The “real-time insulin sensitivity adapted insulin-to-carb ratio” is a corrected insulin-to-carb ratio, based on the difference between the presupposed insulin-sensitivity (the IS calculated by a practitioner at e.g. the start of the treatment or monitoring) and the real-time insulin-sensitivity (IS calculated based on actual insulin and glucose levels in the subject using the device and method according to the invention). The ratio of both IS values results in a correction value, which is used to calculate the more accurate “real-time insulin sensitivity adapted insulin-to-carb ratio”.

Similarly, the “real-time adapted insulin sensitivity glucose correction factor” is a corrected glucose correction factor, based on the difference between the presupposed insulin-sensitivity (the IS calculated by a practitioner at e.g. the start of the treatment or monitoring) and the real-time insulin-sensitivity (IS calculated based on actual insulin and glucose levels in the subject using the device and method according to the invention). The ratio of both IS values results in a correction value, which is used to calculate the more accurate “real-time adapted insulin sensitivity glucose correction factor”.

The present invention hence provides a test device and method that uses a real-time insulin sensitivity adapted basal rate insulin dose for better serving the actual basal insulin need in a patient in need thereof.

The present invention hence provides a test device and method using the real-time insulin sensitivity to better dose the insulin administration in insulin pump users.

The present invention hence provides a test device and method using the beta-cell function calculated from the blood glucose and blood insulin levels for diagnosing patients and monitoring patients with overweight or metabolic syndrome.

The present invention further provides for a method for calculating the real-time insulin resistance, insulin sensitivity or beta-cell function in a subject, comprising the steps of:
measuring the glucose level in a blood sample of the subject,
measuring the insulin level in a blood sample of the subject, and
calculating the real-time insulin resistance, insulin sensitivity or beta-cell function, based on the measured glucose and insulin levels, using the device according to any one of the embodiments described herein. Preferably, said calculation is done using the HOMA1-IR, HOMA2-IR, or HOMA B %, formulas.

The present invention further provides for a method for determining the amount of insulin needed in a type-I diabetes mellitus patient comprising the steps of:
detecting the glucose level in a blood sample of a T1DM patient,
detecting the insulin level in said sample, and
calculating the amount of insulin needed in said patient, based on the real-time insulin sensitivity from the combined insulin/glucose level measured, together with the fasting or pre-meal glucose level in the patient and the quantity of carbohydrates in the next meal, and the calculated Insulin On Board, preferably using the device according to any one of the embodiments described herein.

Preferably, said calculation is done using:
the patient's insulin to carb ratio, to calculate how much insulin is needed to absorb the carbohydrates from the next meal,
the patient's glucose correction factor to calculate how much insulin is needed to correct the fasting or pre-meal glucose level,

both values being corrected for the patient's real-time insulin resistance.

The present invention also allows for more accurate calculation of Insulin On Board (IOB). Insulin On Board (IOB) is residing in the sub-cutis at the place of the last injection(s). It is the amount of insulin that still has to appear into the blood stream over the next few hours. Rather than relying on the time that has elapsed since the last insulin injection to calculate the IOB, the device will determine this by measuring the concentration of insulin in blood, i.e. based on the period of time and the amount of insulin introduced in the last injection and the currently measured insulin concentration, the device will calculate the Insulin On Board.

To properly determine the amount of insulin needed for the next bolus injection, the determined IOB amount should be subtracted from the bolus injection to avoid over-insulinisation, which may result in hypoglycemia, particularly during the time when an individual is sleeping.

The present invention further provides for a method for diagnosing or determining the disease state of a type-2 diabetes mellitus patient, an obese subject, or a subject with metabolic syndrome comprising the steps of:
measuring the glucose level in a blood sample of the subject,
measuring the insulin level in a blood sample of the subject, and
calculating the insulin-resistance or beta-cell function based on the level of blood glucose and insulin measured, preferably using the device according to any one of the embodiments of the invention described herein,
determining the status of the subject, based on said insulin-resistance or beta-cell function, using the device according to any one of the embodiments described herein.

The present invention further provides for a method for screening a population of subjects for the being pre-diabetic or for the risk of becoming a diabetic subject, comprising the steps of:
measuring the glucose level in a blood sample of the subject,
measuring the insulin level in a blood sample of the subject, and
calculating the real-time insulin resistance, using the device according to any one of the embodiments described herein. Preferably, said calculation is done using the HOMA1-IR, HOMA2-IR, or HOMA B %, or any other suitable formula (see Table 1 above for some frequently used formulae). The present invention further provides for a method for better serving the actual basal insulin need of a subject, comprising the step of measuring a real-time insulin sensitivity adapted basal rate of insulin, using a device according to any one of the embodiments described herein.

The present invention further provides for better dosing the insulin administration in insulin pump users, comprising the step of measuring real-time insulin sensitivity, using a device according to any one of the embodiments described herein.

The present invention further provides a better dosing of insulin in insulin pump users by avoiding over insulinisation. By measuring the circulating concentration of Insulin in blood the device enables a more accurate calculation of the Insulin On Board. The device may thus alert a user of potential hypoglycemia in cases where excessive amounts of insulin are present. For example at bedtime, the user would be in a position to suspend insulin delivery for a few hours when too much Insulin On Board is detected. Alternatively the user may decide to consume additional carbohydrate before going to sleep in order to compensate for any excess residual active insulin within the body, which may otherwise lead to hypoglycemia.

The present invention further provides for a method for diagnosing subjects and monitoring subjects with overweight, pre-diabetes or metabolic syndrome comprising the calculation of the beta-cell function in said subjects calculated from the blood glucose and blood insulin levels, determined using a device according to any one of the embodiments described herein.

Measuring blood glucose and insulin can be done simultaneously in the same sample, or can be done subsequently with an interval of e.g. 1 second or more, 5 seconds, 10 seconds, 15 seconds, 20 seconds, 25 seconds, 30 seconds or more, 1 minute, 2, 3, 4, or 5 minutes or more, 10 minutes or slightly more than 10 minutes.

### BRIEF DESCRIPTION OF THE FIGURES

The present invention is illustrated by the following figures which are to be considered for illustrative purposes only and in no way limit the invention to the embodiments disclosed therein:

FIG. 1: Artistic impression of the test device according to the invention. a) The device (1) encompasses a casing (2) with a user interface (3) displaying e.g. the glucose and insulin level measured and a calculated value such as a measure for insulin resistance insulin sensitivity or beta-cell function, and a keypad (4) to allow the user to process the data retrieved or e.g. to enter user specific data into the device; a test strip (5) can be entered into the device, e.g. carrying the reagents and the blood sample. b) Artistic impression of the device when suggesting a bolus amount of insulin.

FIG. 2: Flow-chart of how the test works for type-II diabetes mellitus patients, obese subjects or subjects with metabolic syndrome. A blood sample is deposited on the test strip, which is brought into contact with the test device. The test device measures the blood glucose and insulin level in said blood sample and calculates the insulin resistance, using a HOMA-IR formula, and/or the beta-cell function using the HOMA-B % formula. Other formulae as described in Table 1 may also be implemented to determine either insulin resistance and/or beta cell function. The result can be displayed to the user (patient or healthcare practitioner) who can e.g. save the data for future reference e.g. for comparing insulin-resistance and/or beta-cell function before and after exercise or for monitoring the disease development, and/or the effect of a treatment. The user can also interact with the device to e.g. enter the date and time of the measurement.

FIG. 3: Flow-chart of how the test works for type-I diabetes mellitus patients. A blood sample is deposited on the test strip, which is placed in the test device. The test device measures the blood glucose and insulin level in said sample and calculates the insulin sensitivity (1/insulin resistance). The user can interact with the device to enter the amount of carbohydrates in the meal to be digested and the target glucose level to be achieved by the user. The device then calculates the real-time insulin adapted glucose correction factor and real-time insulin adapted insulin to carb ratio. The device can also calculate the Insulin On Board based on the amount and time of a previous insulin injection(s) and the current concentration of insulin measured in a sample of blood obtained from the patient. The device determines the amount of insulin required to accommodate the carbohydrate load in the next meal to be consumed and thereby seeks to bring the fasting glucose level to the target level. The calculated Insulin On Board is used to determine the required dose of insulin and the next bolus amount is displayed. The user can also interact with the device to e.g. enter the date and time of the measurement.

FIG. 4: Schematic representation of an exemplary disposable test strip for detecting glucose and insulin in a single drop of blood. This schematic represents a disposable test strip (5), comprising a sample receiving means (501), which is capable of distributing the sample into multiple microfluidic channels (502 to 503), for simultaneous detection of blood glucose level and insulin level. Each channel is accompanied with a pair of electrodes, a working electrode (508) and a counter/reference electrode (509). The test strip has four zones: a sample receiving zone (510), a sample distribution zone (511), a reaction zone (512) and an analyte detection zone (513). Each working electrode has a certain output signal (502a to 503a) and each counter/reference electrode has a certain output signal (502b to 503b), which can be read by a controlling device, designed to be in contact with said different electrodes and that can control the operation of the device and analyse the data obtained from the biosensor system. The number of channels is not to be seen as limited to the 2 channels represented herein, but may include more channels according to the function of the device.

FIG. 5: Schematic representation of an exemplary glucose detecting sensor on one microfluidic channel of the test strip. a) The sample comprising glucose (603), is directed towards the sample reaction zone (512) through capillary force. b) In the reaction zone (512), glucose is oxidized (603′) by a suitable oxidoreductase enzyme, for example glucose oxidase or glucose dehydrogenase (601), which is present in reaction zone (512). Said oxidation process releases electrons, which are transferred to the working electrode, e.g. by means of a suitable electron mediator (602).

The number of electrons liberated during the oxidation of glucose by the oxidoreductase enzyme system is proportional to the amount of glucose present in the sample and is measured as an output signal (502a*).

FIG. 6: Schematic representation of an exemplary insulin detecting sensor on another microfluidic cannel of the test strip. a) The sample comprising insulin (703), is directed towards the sample reaction zone (512) through capillary force. b) In the reaction zone (512), the insulin is bound by two antibodies: a first antibody, complexed with an enzyme label (701), and a second antibody, complexed with a magnetic particle (702), both present in the reaction zone. Upon metabolizing its substrate (704), present at the detection zone, the enzyme label (701) will generate an electrochemical signal, i.e. releasing one or more electrons, which are detected by the working electrode (508), placed in the detection zone. c) Outside the detection zone (513), e.g. in the reaction zone (512), a magnet (514) can be placed, which upon activation (514*), will draw away all magnetic bead-second antibody complexes from the detection zone. When insulin is present, it will be bound to the second antibody-magnetic bead and will hence be attracted to the magnet as well, together with the first antibody-enzyme complex. This reduces the amount of electrons produced at the site of the working electrode (508) and detection zone (513). Both signals 503a and 503a* can be detected by a reader. The difference in number of electrons formed at the working electrode before and after activation of the magnet is proportional to the amount of insulin in the sample. d) Alternatively, the magnet (514′) can be situated at the working electrode (508) in the detection zone (513). e) When activated (514′*) said magnet can now attract the second antibody-magnetic bead complexes to generate electrons at the working electrode (508) where the substrate (704) is present in an amount proportional to the amount of insulin present in the sample. Such an assay may include a step to eliminate the non-bound enzyme label in order to increase the sensitivity and accuracy.

FIG. 7: Measurement of insulin (C-peptide) in 5 microliter whole blood samples from healthy subjects (n=6). The blood samples were spiked with a known concentration of C-peptide, indicating the measurements are accurate in a range of 0 to 10.000 pM, using the test device of FIGS. 4 and 6 (cf. Example 1).

FIG. 8: Measurement of glucose in 5 microliter whole blood samples (n=6, same subjects as in FIG. 7), using the test device of FIGS. 4 and 5 (cf. Example 1).

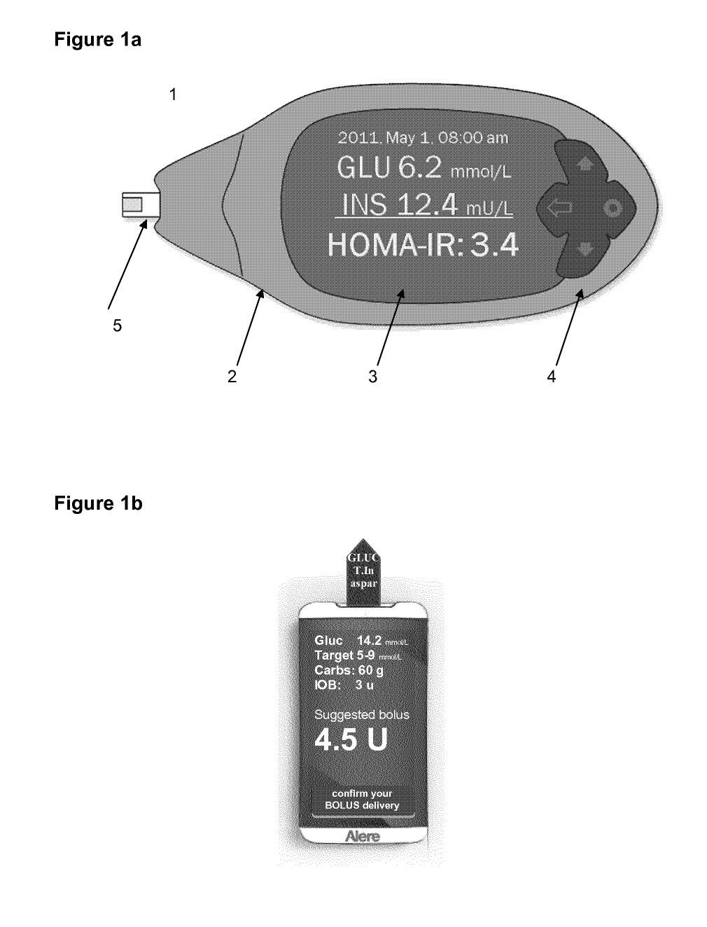

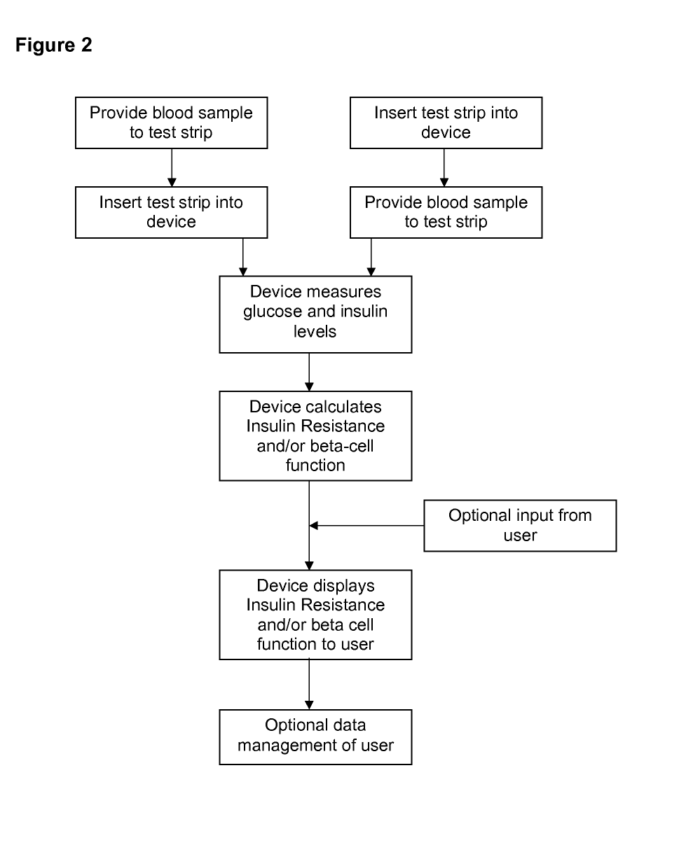

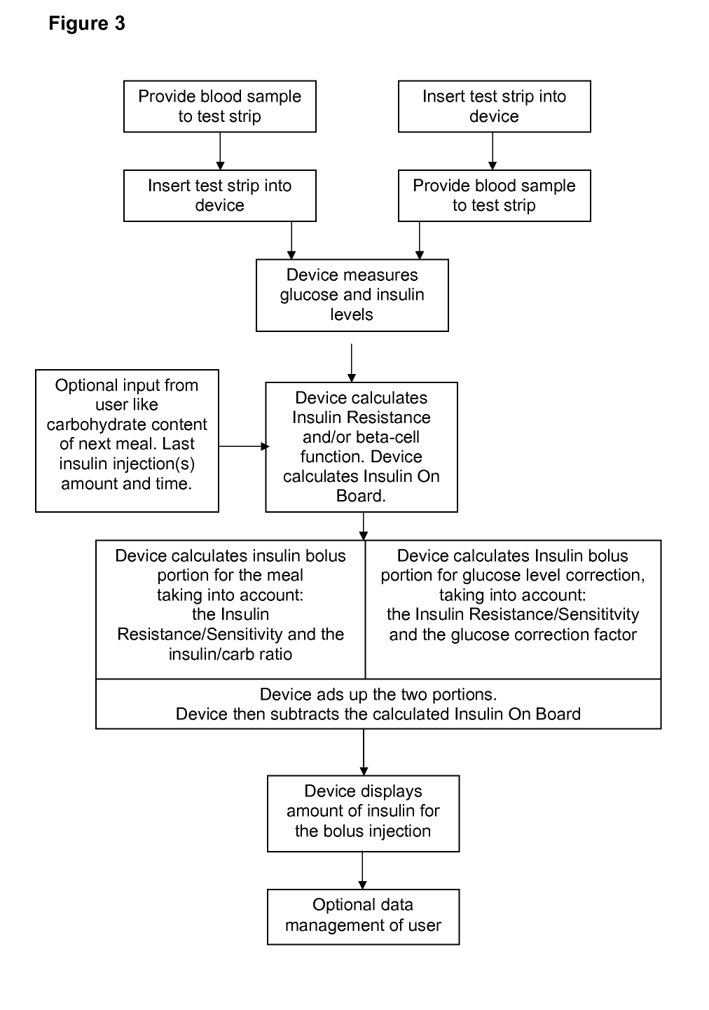

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

The recitation of numerical ranges by endpoints includes all numbers and fractions subsumed within the respective ranges, as well as the recited endpoints.

The term “about” as used herein when referring to a measurable value such as a level, an amount, a parameter, a temporal duration, and the like, is meant to encompass variations of and from the specified value, in particular variations of +1-10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The methods and devices disclosed herein can be supplemented with the analysis of further (bio)markers that are useful for the diagnosis, prediction, prognosis and/or monitoring of the diseases and conditions as disclosed herein. By means of example and not limitation, biomarkers useful in evaluating beta-cell function and insulin resistance include for example VMAT2, which is an indicator of beta cell mass, free fatty acid level (FFA's), and magnetic nanoparticle effusion technique (cf. Martz, L. SciBX 3(48); doi:10.1038/scibx.2010.1433) can also be used to evaluate residual beta-cell activity in a T1DM, or T2DM subject. These can be combined with the measurements made by the device and method according to the present invention.

The terms “predicting” or “prediction”, “diagnosing” or “diagnosis” and “prognosticating” or “prognosis” are commonplace and well-understood in medical and clinical practice. It shall be understood that the phrase “a method for the diagnosis, prediction and/or prognosis” a given disease or condition may also be interchanged with phrases such as “a method for diagnosing, predicting and/or prognosticating” of said disease or condition or “a method for making (or determining or establishing) the diagnosis, prediction and/or prognosis” of said disease or condition, or the like.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of” the diseases or conditions as taught herein in a subject may particularly mean that the subject has such, hence, is diagnosed as having such. “Diagnosis of no” diseases or conditions as taught herein in a subject may particularly mean that the subject does not have such, hence, is diagnosed as not having such. A subject may be diagnosed as not having such despite displaying one or more conventional symptoms or signs reminiscent of such.

The terms “prognosticating” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

By means of further explanation and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require to first determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need to first convert the readouts to absolute values of the respective parameters.

The term “real-time” as used herein indicates that the insulin-resistance was measured recently, e.g. approximately within the last 24, 12, or 6 hours, or is the insulin resistance measured at the time of preparing the bolus-injection. It in fact indicates that both blood glucose and insulin levels have been detected at substantially the same moment, resulting in a real-time insulin resistance or sensitivity, rather than based on insulin values that were measured weeks or months ago in the practitioner's office. Measuring blood glucose and insulin can be done simultaneously in the same sample, or can be done substantially at the same moment i.e. with an interval of e.g. 1, or a few seconds, 5 seconds, 10 seconds, 15 seconds, 20 seconds, 25 seconds, 30 seconds or more, 1 minute, 2, 3, 4, or 5 minutes or more, 10 minutes or slightly more than 10 minutes.

The term “insulin” as used herein encompasses all detectable forms and fragments of insulin and can be produced by the subject (endogenous) or can have been administered exogenously.

In the beta cells within islets of Langerhans of the pancreas, insulin is originally produced as a single molecule, called pre-pro-insulin, composed of 110 amino acids. After this has passed through the endoplasmic reticulum, 24 amino acids (“the signal peptide”) are removed by enzyme action from one end of the chain, resulting in pro-insulin, which folds and bonds to give the molecule almost it's final structure. This passes into vesicles budded off from the Golgi body. Here a middle section, the “C chain” or “C-peptide” of 33 amino-acids is removed by the action of the enzymes pro-hormone convertase 1 and 2, converting it into the final structure with 2 chains, A and B, and 2 amino acids are then removed by another enzyme carboxypeptidase E. The final three-dimensional structure of insulin is then further stabilised by disulphide bridges. These form between thiol groups (—SH) on cysteine residues (CYS above). There are 6 cysteines, so 3 disulphide bridges are formed: 2 between the A and B chains, and one within the A chain.

The C-peptide level in blood hence reflects the amount of insulin that was totally produced by the subject. This is in contrast to the level of mature insulin in the blood, since it first passes through the liver, where a significant part is metabolised in a variable way. The peripheral (e.g. in a forearm or a finger stick drop) blood level of endogenous insulin is hence not exactly representing the beta-cell activity. C-peptide is only removed from the blood by the kidneys and is not used and metabolised by the liver. Therefore, peripheral blood levels of C-peptide reflect better the beta-cell function than peripheral insulin levels.

In addition to the endogenous insulin and the cleaved C-peptide part thereof, also the exogenously administered insulin in certain patient types may be detectable. Insulin can hence be detected using a general antibody or a mixture thereof, which will measure the total amount of insulin (i.e. endogenous plus exogenous) in the blood of the patient. In the alternative, specifically measuring C-peptide levels in blood will reflect the actual endogenously produced insulin in the patient and hence reflect the beta-cell activity. The exogenous insulin can be administered in basically four formats:
1. Human insulin that appears in the blood undistinguishable from the endogenous insulin
2. Insulins that have been recombinantly modified and are hence also distinguishable using specific antibodies directed to the modified amino acids. One such a recombinant is a extra-short and fast working insulin such as: Humalog (Lispro), NovoLog (Aspart), Apidra (Glulisine)
3. another recombinant form is the extra long working insulins such as: Lantus (Glargine).
4. Levemir (Detemir) is an insulin where a fatty acid chain is bound to to prolong its half-life in the sub-cutis. The slower release makes it a long acting insulin. Once it enters the blood stream, it becomes indistinguishable from human insulin.

Any combination of the insulins above can be used in one patient. One can therefore decide to measure all types using a single general antibody-pair, or one can decide to detect the amount of long- or short-acting insulin separately, depending on the condition or disease state of the subject. Some examples can be:
“rapid-onset insulin” or “fast-acting insulin” has a peak time of about one hour and lasting for three to five hours. This type of insulin is typically used directly before eating: the bolus insulin.
“short acting insulin” begins to lower blood glucose levels within 30 minutes, so need to be administered half an hour before eating. It has peak effect of four hours and works for about six hours.
“Intermediate acting insulin” has either protamine or zinc added to delay their action. This human insulin starts to show its effect about 90 minutes after injection, has a peak at 4 to 12 hours, and lasts for 16 to 24 hours.
“Mixed insulin” is a combination of either a rapid onset-fast acting or a short acting insulin and intermediate acting insulin. Advantage of it is that, two types of insulin can be given in one injection. When it shows 30/70 then it means 30% of short acting is mixed with 70% of intermediate acting insulin.
“Long acting insulin” There are two kinds of long acting insulin available in market: Lantus (Glargine)—It has no peak period as it works constantly when released into your bloodstream at a relatively constant rate. (full 24 hours) and Levemir (Detemir)—It has a relatively flat action, can last up to 24 hours and may be given once or twice during the day.

The table below provides some exemplary but non-limiting insulins that are suitable for treating patients with diabetes and that could be measured using the device and method according to the present invention:

| Types of Insulin | Examples | Onset of Action | | Peak of Action | | Duration of Action | |
|---|---|---|---|---|---|---|---|
| Rapid-acting | Humalog (lispro) | 15 | minutes | 30-90 | minutes | 3-5 | hours |
| | Eli Lilly | | | | | | |
| | NovoLog (aspart) | 15 | minutes | 40-50 | minutes | 3-5 | hours |
| | Novo Nordisk | | | | | | |
| Short-acting | Humulin R | 30-60 | minutes | 50-120 | minutes | 5-8 | hours |
| (Regular) | Eli Lilly | | | | | | |
| | Novolin R | | | | | | |
| | Novo Nordisk | | | | | | |
| Intermediate- | Humulin N | 1-3 | hours | 8 | hours | 20 | hours |
| acting (NPH) | Eli Lilly | | | | | | |
| | Novolin N | | | | | | |
| | Novo Nordisk | | | | | | |
| | Humulin L | 1-2.5 | hours | 7-15 | hours | 18-24 | hours |
| | Eli Lilly | | | | | | |
| | Novolin L | | | | | | |
| | Novo Nordisk | | | | | | |
| Mixed acting | Humulin 50/50 | The onset, | | | | | |
| | Humulin 70/30 | peak, and | | | | | |
| | Humalog Mix | duration of | | | | | |
| | 75/25 | action of these | | | | | |
| | Humalog Mix | mixtures would | | | | | |
| | 50/50 | reflect a | | | | | |
| | Eli Lilly | composite of | | | | | |
| | Novolin 70/30 | the | | | | | |
| | Novolog Mix | intermediate | | | | | |
| | 70/30 | and short- or | | | | | |
| | Novo Nordisk | rapid-acting | | | | | |
| | | components, | | | | | |
| | | with one peak | | | | | |
| | | of action. | | | | | |
| Long-acting | Ultralente | 4-8 | hours | 8-12 | hours | 36 | hours |
| | Eli Lilly | | | | | | |

| | | | | | |
|---|---|---|---|---|---|
| Lantus (glargine) | 1 | hour | None | 24 | hours |
| Aventis | | | | | |

It is important in certain situations to know the origin of low or high glucose levels in a patient. By measuring the different types of insulin one may identify the specific problem. Without measuring the different types, it is difficult to know which kind of insulin dose to adjust.

### The Testing Device

The present invention provides test devices for the diagnosis, prediction, prognosis and/or monitoring of any one disease or condition as taught herein comprising means for detecting the level of glucose and insulin in a blood or serum sample of the patient. In a more preferred embodiment, such device of the invention can be used in clinical settings or at home. The device according to the invention can be used for diagnosing said metabolic disease or condition as defined herein, for monitoring the effectiveness of treatment of a subject suffering from said disease or condition with an agent, or for preventive screening of subjects for the occurrence of said disease or condition in said subject.

The device can be in the form of a home test device or a point of care device (POC). The device can assist a medical practitioner, or nurse to decide whether the patient under observation is developing a disease or condition as taught herein, after which appropriate action or treatment can be performed.

The device can e.g. assist a subject having diabetes to control or fine-tune the amount of insulin needed during the day or before a meal or allows him to monitor his insulin resistance or sensitivity throughout the day, e.g. in function of the physical state or condition the subject is in.

The device can further assist in motivating an obese subject or a subject with metabolic syndrome or a person with pre-diabetes to perform the necessary exercises, by following the insulin resistance value before and after training.

Typical devices according to the invention comprise a means for measuring the amount or level of both glucose and insulin in a blood sample, visualizing the amount of glucose and insulin in said sample and indicating the insulin resistance and/or sensitivity of the subject at that moment.

In a preferred embodiment, the invention provides a lateral flow device. Such lateral flow device comprises a test strip allowing migration of a sample by capillary flow from one end of the strip where the sample is applied to the other end of such strip where presence of an analyte in said sample is measured. In another embodiment, the invention provides a device comprising a reagent strip, encompassing a reaction zone which will yield a quantitative signal upon interaction with the analyte. This signal can be generated by electrochemical or optical/photometric systems.

A “binding molecule” as intended herein is any substance that binds specifically to a marker. Examples of a binding molecule useful according to the present invention, include, but are not limited to an antibody, an antibody fragment, a polypeptide, a peptide, a lipid, a carbohydrate, a nucleic acid (aptamer, spiegelmer), peptide-nucleic acid, small molecule, small organic molecule, or other drug candidate.

According to an aspect of the invention, a “binding molecule” preferably binds specifically to said one or more markers with an affinity of at least, or better than 10⁻⁶ M. A suitable binding molecule can be determined from its binding with a standard sample of said one or more markers. Methods for determining the binding between binding molecule and said any one or more markers are known in the art. As used herein, the term antibody includes, but is not limited to, polyclonal antibodies, monoclonal antibodies, humanised or chimeric antibodies, engineered antibodies, and biologically functional antibody fragments (e.g. scFv, nanobodies, Fv, etc) sufficient for binding of the antibody fragment to the protein. Such antibody may be commercially available antibody against said one or more markers, such as, for example, a mouse, rat, human or humanised polyclonal or monoclonal antibody.

### Electrochemical Analyte Detection

In currently available home tests or POC tests, the blood glucose level is typically measured using electrochemical detection methods. Many glucose meters employ the oxidation of glucose to gluconolactone catalyzed by glucose oxidase or glucose dehydrogenase.

Test strips typically contain a capillary channel that adsorbs a reproducible amount of the blood sample. The glucose in the blood reacts with an enzyme electrode containing glucose oxidase or dehydrogenase and the enzyme is oxidized with an excess of an electron-mediator. The mediator in turn is oxidised by reaction at the electrode, which generates an electrical current. The total charge passing through the electrode is proportional to the amount of glucose in the blood that has reacted with the enzyme. There are two ways of analysing the charge yielded: a coulometric method (total amount of charge generated by the glucose oxidation reaction over a period of time), or an amperometric method (measures the electrical current generated at a specific point in time by the glucose reaction). The coulometric method can have variable test times, whereas the test time on a meter using the amperometric method is fixed. Both methods give an estimation of the concentration of glucose in the blood sample.

In essence, the amount of glucose is detected by measuring the charge yielded between two tiny electrodes, which can e.g. be printed on a disposable test strip to which a drop of blood of the subject is added. One of these electrodes encompasses an amount of the glucose oxidase or dehydrogenase enzyme and a certain amount of electron transfer mediator. The glucose present in the blood drop is oxidized by the oxidase or dehydrogenase, which releases (an) electron(s) proportionate to the amount of glucose that is present in the sample. These electrons are then transferred to the second electrode and the current is measured by a simple charge (Volt-Ampero)-meter, and the amount of measured electrons is then extrapolated to the blood glucose level of the subject doing the test.

Insulin blood level home tests or POC tests are to our knowledge not yet available. One possible test device according to the present invention detects insulin based on an electrochemical immunoassay detection system.

In essence, any electrochemical system can be used. One example is to label the analyte-specific antibody with any charged molecule or particle.

Preferred examples could be metal particles such as Al3+, Ag+, Au3+, Cu2+, and the like. Non-magnetic particles may be preferred for reasons set out below. The antibody-analyte complexes can then be detected by using a second antibody specific for the analyte, which can e.g. be fixed to an analyte detection zone on the test strip, or which is attracted to said zone by other means such as e.g. magnetism (see below). The analyte detection zone comprises a set of 2 or 3 electrodes, two opposite charged electrodes forming an electrode couple and optionally a reference electrode in the middle of said couple. The now fixed antibody-analyte-antibody-charged-label complex is then directed to an opposite charged electrode by inducing a charge or electric current between both electrodes. The antibody-analyte complexes are now attracted to the opposite charged electrode (e.g. positive charged particles will be attracted to the negative pole of the electrode couple). The charge or current is then reversed, thereby releasing the complexes and moving them to the opposite electrode and the current resulting from this change is measured. The measured total current received at the second electrode or at the reference electrode is proportional to the amount of complex that was displaced from the first electrode. In between the two working electrodes, a reference electrode may be placed, in order to simplify the distinction between the induced current and the current caused by the displacement of the labeled antibody-analyte complexes.

In said embodiments, the charged particle-antibody-analyte complex can be attracted to the reaction zone by using a second antibody which carries a magnetic particle. Inducing magnetism at the reaction zone will attract all second-antibody-antigen-antibody-charged-label complexes and the non-bound reagents will no longer interact with the test.

An alternative solution is the use of an enzyme-activation electrochemical detection system such as the one disclosed in U.S. Pat. No. 7,166,208, hereby incorporated by reference. In essence, the system encompasses a fixed enzyme, which releases electrons upon binding of the substrate (e.g. apoglucose oxidase). Said substrate is linked to an antibody which is specific for the insulin analyte to be measured. Said substrate is however also modified such that it will only bind to its enzyme, when an analyte is attached thereto. In this system, the enzyme thus only releases electrons when bound by a substrate-antibody-analyte complex and the electron current measured on the second electrode is again proportional to the amount of analyte (in this case insulin) present.

In an alternative form, an electron-releasing enzyme system can be coupled to an analyte specific antibody. Secondary analyte-specific antibodies, linked to magnetic beads, can help in sequestering only analyte-bound enzyme-complexes. Upon contact with its substrate, an electron is formed by said enzyme and the current obtained through said enzymatic activity is measured. The system can of course be reversed, wherein the magnetic beads can also be used to capture away the enzyme-analyte complexes, wherein a reduction of electronic current initially present will be proportional to the analyte presence.

In essence, any form of electrochemical detection of insulin can be used. Below, some non-limiting examples are discussed, but any alternative system may be equally useful.

The device and method according of the present invention can make use of enzyme-linked immunomagnetic electrochemistry (ELIME), which combines the enzymatic oxidation-reduction (yielding an electrochemical “signal”) of a substrate that is bound to an analyte-specific antibody, with a second analyte-specific antibody which is linked to a magnetic particle and concentrated at the electrode. The principle of ELIME can e.g. be read in Gehring and Tu, 2005 (J. of Food Protection Vol. 68(1):146-149 and U.S. Pat. No. 6,682,648.

Apart from ELIME, the principle of immunomagnetic detection of an analyte in a sample can also be used independently such as in the Magnotech sensor from Phillips. In such as sensor, magnetically labeled antibodies, specific for the analyte are used to trap said analyte. Secondary analyte-specific antibodies are fixed to the substrate of the sensor. Upon magnetizing the substrate, the magnetically labeled antibody-analyte complexes are drawn towards the substrate, where they can now bind to the secondary antibodies. After that, the magnetic field is reversed, releasing all unbound labeled antibodies. The amount of bound labeled antibodies is indicative for the amount of analyte present and can then be measured using light diffraction, scattering or reflection caused by said magnetic beads. The Magnotech sensor is capable of detecting picomolar amounts of BNP or Troponin-1 in a blood sample. Other examples of commercially available sensors are the Alere Heart-check and EPOcal systems.

The device and method according of the present invention can also make use of an electrochemical immunoassay system such as the one exemplified in U.S. Pat. No. 5,391,272.

Another electrobiochemical system that can be used in the device and method according of the present invention is disclosed in U.S. Pat. No. 5,942,388, describing an electrobiochemical system comprising an electrode having immobilized thereon a member of a recognition pair, the other member of said pair being said analyte, the presence of said analyte in the medium resulting in formation of a pair complex, being a complex between said immobilized member and said analyte; the system further comprising redox molecules capable of changing their redox state by accepting electrons from or donating electrons to the electrode; the formation of the pair complex on the electrode bringing a change in the electrical response of the system, whereby the presence and optionally the concentration of said analyte in the medium can be determined.

Alternatively, the device and method according of the present invention can make use of an eletrochemical alkaline phosphatase immunoassay comprising the steps of contacting the alkaline phosphatase with 1-naphthyl phosphate, allowing the phosphatase to hydrolyse the 1-naphthyl phosphate to form 1-naphthol and detecting the electrochemical oxidation potential of said 1-naphthol using an electrode comprising resin bonded particles of carbon having a particle size of 3 to 50 nm the particles carrying a platinum group metal.

In yet an alternative embodiment, the device and method according of the present invention can use an electrochemical detection system based on a horseradish peroxidase enzyme immunoassay using p-aminophenol as substrate, such as e.g. the assay described in Wei Sun et al., 2001, Analytica Chimica Acta 434:43-50.

Alternatively, the device and method of the present invention can make use of enzyme-linked immunomagnetic chemiluminescence (ELIMCL) such as referred to in e.g. Gehring et al., 2004, J. Immunological Methods, Vol 293:97-106.

The test device of the invention can in another embodiment also use carbon nanotube based immunosensors as disclosed e.g. in US20060240492A1. In essence, these detector devices use a carbon-based nanotube that acts as an electrode. In stead of generating a signal “above” the electrode, the signal and electrochemical reaction is generated inside the electrode and then transferred to a charge or current measuring system.

Another exemplary technology is that of a piezo-electric based sensor such as the ones developed by Vivacta (for TSH detection. In said sensor, an analyte-specific primary antibody is fixed on the surface of a piezofilm. Secondary antibodies coated with carbon particles in solution are also able to bind to the analyte, trapped by the primary antibody. A LED pulse is then fired at the film creating heating of the carbon particles on said piezo-electric film, which deforms it slightly, producing an electric charge. The amount of charge produced is a measure for the amount of carbon particles trapped by the film and hence of the analyte concentration in the sample. This sensor only uses a minor drop of blood (e.g. from a finger prick) and can detect TSH in picomolar amounts, without the need of filtering or washing steps.

The “electron transfer mediator” used in the devices of the present invention is preferably selected from the group consisting of hexaamineruthenium (III) chloride, a ferricyanide ion such as potassium ferricyanide, potassium ferrocyanide, dimethylferrocene, ferricinium, a ferrocene derivative, phenoxazine derivatives, phenothiazine derivatives, quinone derivatives, and reversible redox transition metal complexes, particularly those of Ruthenium and Osmium, nicotinamide adenine dinucleotide (phosphate), diimines, phenanthroline derivatives, dichlorophenolindophenol, tetrazolium dyes, ferocene-monocarboxylic acid, 7,7,8,8-tetracyanoquinodimethane, tetrathiafulvalene, nickelocene, N-methylacidinium, tetrathiatetracene, N-methylphenazinium, hydroquinone, 3-dimethylaminobenzoic acid, 3-methyl-2-benzothiozolinone hydrazone, 2-methoxy-4-allylphenol, 4-aminoantipyrin, dimethylaniline, 4-aminoantipyrene, 4-methoxynaphthol, 3,3,5,5-tetramethylbenzidine, 2,2-azino-di-[3-ethylbenzthiazoline sulfonate], o-dianisidine, o-toluidine, 2,4-dichloro phenol, 4-aminophenazone, benzidine, Prussian blue, hydrogenperoxide, or an osmium bipyridyl complex, or any other elector transfer mediator known in the art.

### Colorimetric/Photometric Analyte Detection

In the alternative, a colorimetric signal can be detected, which is proportional to the amount of analyte present in the sample. In essence, any enzymatic or other chemical reaction yielding a visually detectable signal (colour, turbidity, fluorescence, etc.) can be used. Typically, such sensors actually measure the amount of substrate that is converted by a specific enzyme. In some case the substrate is the actual analyte to be detected (e.g. in case of glucose) in other systems, a more complex chain-reaction of masking and unmasking of enzymes is triggered upon the presence of the analyte. These typically employ immunology-based triggers, wherein in the presence of the analyte, a specific binding partner (e.g. an antibody) changes its confirmation and hence can trigger or activate the activity of an enzyme, which in turn reacts with its substrate, yielding a colored of visually detectable complex.

Alternatively, the detection is based on pure immunological techniques, which in fact employ standard ELISA technology with deposited analyte-specific antibodies, incorporated on a micro-scale in the reaction zone of a test strip. The lateral or capillary flow present in such test strips is generally sufficient to drive the analyte over the reaction zone, where it is bound to the specific binding agent or antibody. Bound analytes are then detected by other labeled antibodies binding the trapped analyte complexes. The fluid present in blood in combination with the capillary forces can already act as a “washing” step of unbound and hence unwanted contaminants. In some cases, a small reservoir of liquid is linked to the test strip, to improve the washing step. The labeled antibody-analyte-antibody complex can then be detected by standard colorimetric optics, illuminating on or through said reaction zone. The amount of bound-complexes will determine the amount of analyte present in the sample.

Colorimetric tests comprise optics to illuminate the reaction zone on said test strip and detect a colorimetric (reflection, transluminescence, absorption of light, fluorescence etc.) property thereof, which is then digitized in order to calculate the amount of analyte in the sample deposited on the test strip.

Examples of blood glucose tests are well known and use the same colorimetric reaction that is still used nowadays in glucose test strips. For example, Urine glucose strips use glucose oxidase, and a benzidine derivative, which is oxidized to form a blue-colour polymer by the hydrogen peroxide formed in the oxidation reaction. Alternatively, the GOD-Perid method can be used, wherein test strips comprise an amount of peroxidase enzyme, which will convert ABTS into a colored complex in the presence of hydrogen peroxide. Since this hydrogen peroxide is again formed upon reaction of glucose oxidase with blood glucose, the amount of colored complex formed is again proportional to the amount of glucose present in the blood sample.

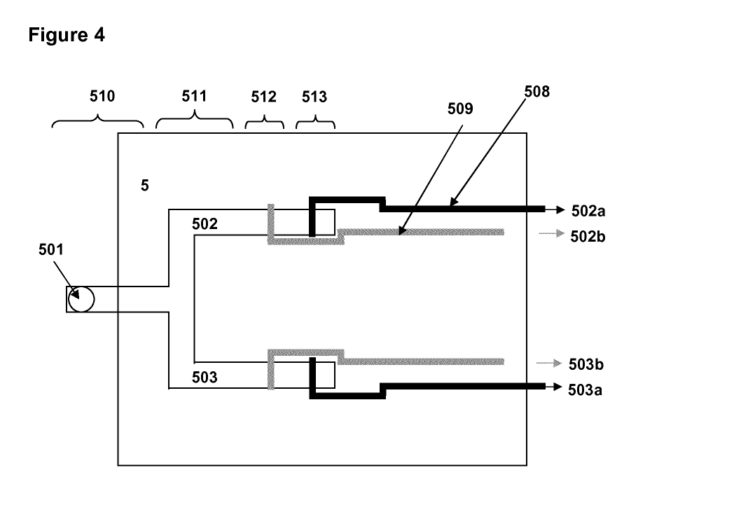

In a preferred embodiment of the testing device of the present invention, detecting both glucose and insulin levels in a blood sample of a subject comprises a disposable test strip which can receive a drop of blood. Said strip preferably comprises a) a sample receiving part; and b) an analyte reaction zone comprising: b1) a first electrochemical or optical sensor for detecting the blood glucose level in said sample, and b2) a second electrochemical or optical sensor for detecting the blood insulin level in said sample. The sample is directed to the different zones through multiple microfluidic channels on the strip. The testing device further comprises c) a controlling device that can control the operation of the device and analyse the data obtained from the biosensor systems; and d) a user interface, displaying the data to the user. The schematic in FIG. 4 represents an exemplary disposable test strip (5), comprising a sample receiving means (501), which is capable of distributing the sample into two or more multiple microfluidic channels (502 to 503), for simultaneous detection of blood glucose level (e.g. 502) and insulin level (503). Each channel is equipped with a pair of electrodes, a working electrode (508) and a counter/reference electrode (509). The test strip comprises four zones: a sample receiving zone (510), a sample distribution zone (511), a reaction zone (512) and an analyte detection zone (513). Each working electrode has a certain output signal (502a and 503a) and each counter/reference electrode has a certain output signal (502b and 503b), which can be read by a controlling device, designed to be in contact with said different electrodes and that can control the operation of the device and analyse the data obtained from the biosensor systems. The number of channels is not to be seen as limited to the 2 channels represented by the exemplary embodiment described herein with respect to FIG. 4. In principle, two channels will suffice, since two analytes, namely glucose and insulin need to be detected. Other channels can be supplied for detecting other interesting blood analytes, or can be used as control channels, or to permit multiple measurements e.g. in different concentration ranges of the same analyte. Multiple measurements of glucose and insulin can be made in multiple channels, in order to reduce the error margin and increase the accuracy of the measurements.

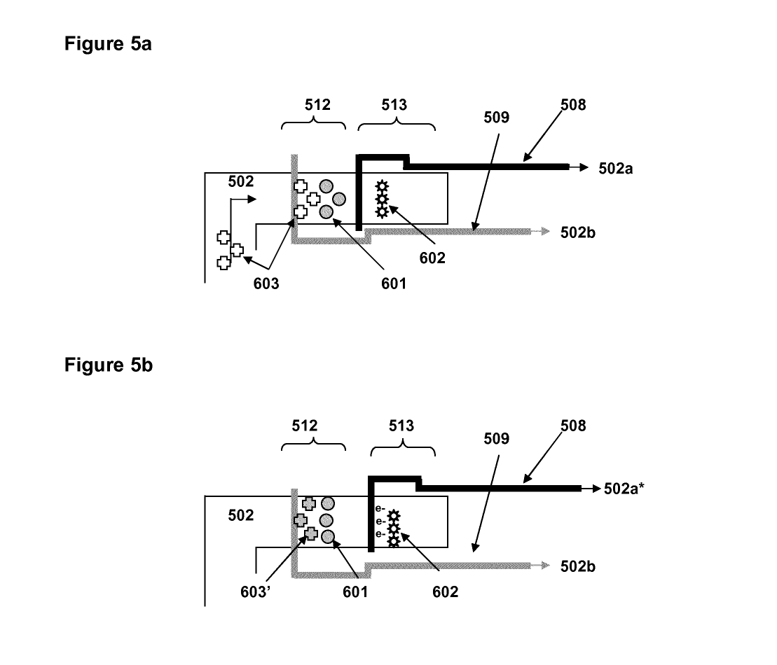

In a preferred embodiment said first sensor b1) (e.g. 502 in FIGS. 4 and 5) for detecting glucose typically comprises a screen printed working and counter/reference electrode on the disposable test strip. To the working electrode, an amount of oxidoreductase, such as glucose oxidase or glucose dehydrogenase is attached, in combination with an amount of electron-transfer mediator. The glucose in the blood sample brought onto the test strip is oxidized by the oxidoreductase present on the working electrode, thereby releasing a proportional amount of electrons, transferred by the mediator to the counter/reference electrode. The current measured between both electrodes is proportional to the amount of glucose in the blood sample. FIG. 5 exemplifies this process: a) The sample comprising glucose (603), is directed towards the sample reaction zone (512) through capillary force. b) In the reaction zone, it is oxidized (603′) by glucose oxidase (601) present in the reaction zone. Said oxidation process releases electrons, which are transferred to the working electrode, e.g. by means of an electron mediator (602). The electron production of the glucose oxidase system is proportional to the amount of glucose present in the sample and is measured as an output signal (502a*).

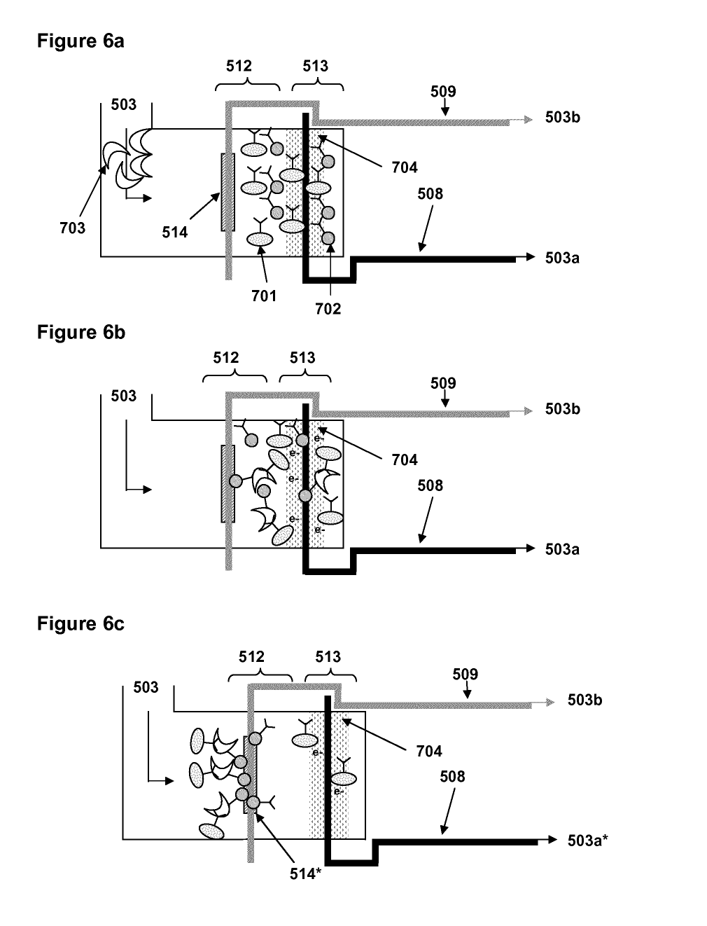

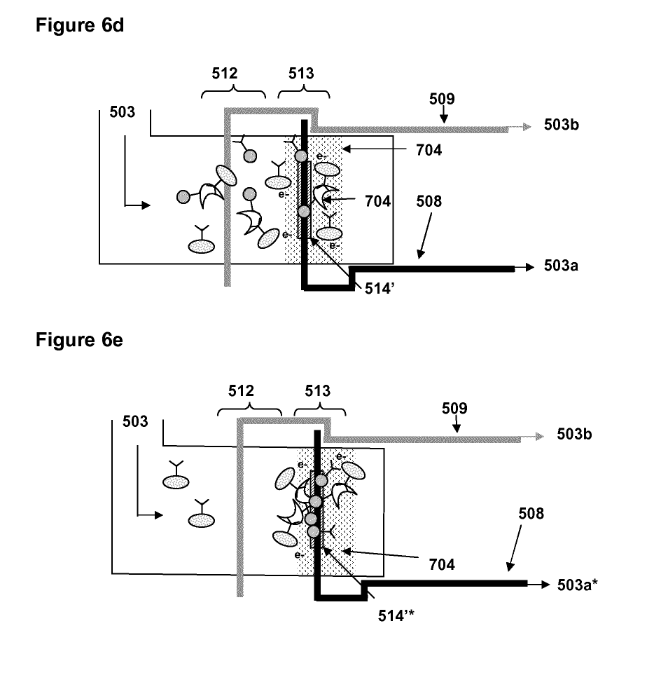

In a preferred embodiment said second sensor b2) (e.g. 503 in FIGS. 4 and 6) for detecting insulin is an electrochemical sensor, measuring a change in charge or current due to enzymatic reaction with a substrate upon binding of insulin, more particularly an enzyme-linked immunomagnetic electrochemical assay. Said assay comprises: an electron-releasing enzyme system coupled to an insulin-specific antibody and secondary insulin-specific antibodies, linked to magnetic particles.

Upon contact with its substrate, an electron is formed by said enzyme and the current obtained through said enzymatic activity is measured. In the presence of an electron transfer mediator the electron-transfer mediated by the enzyme complex is monitored using for example a screen printed working (and counter/reference) electrode on the disposable test strip.

In order to avoid any washing steps, magnetic particles, linked to the second anti-insulin antibodies, are used to withdraw any insulin-bound enzyme complexes (complexed through a first anti-insulin antibody). The subsequent reduction in current signal generated at the working electrode versus the initial current signal prior to withdrawal of magnetic particle/insulin complexes is proportional to the amount of insulin present in the sample. FIG. 6 exemplifies this process: a) The sample comprising insulin (703), is directed towards the sample reaction zone (512) through capillary force. b) In the reaction zone, the insulin is bound by two antibodies: a first antibody, complexed with an enzyme label (701), and a second antibody, complexed with a magnetic particle (702), both present in the reaction zone. The enzyme will produce electrons upon metabolizing its substrate (704), present in the detection zone, which in the presence of an electron mediator, will be detected by the working electrode (508), placed in the detection zone. c) Outside the detection zone (513), e.g. in the reaction zone (512), a magnet (514) is placed, which upon activation (514*), will draw away all magnetic bead-second antibody complexes from the detection zone. When insulin is present, antibody-magnetic particle-insulin will form. Such complexes are susceptible to a localised magnetic field, and as such will be attracted to the magnet (514) along with any of the first antibody-enzyme complex that has formed “sandwich” complexes with the target, insulin. Removal of first antibody-enzyme complexes from the reaction zone (512) leads to a reduction in reaction between enzyme label and substrate at the working electrode (508). This reduces the amount of electrons produced at the site of the working electrode (508) and detection zone (513). Both signals 503a and 503a* can be detected by a reader. The difference in number of electrons formed at the working electrode before and after activation of the magnet is proportional to the amount of insulin in the sample. The greater the amount or concentration of insulin present in the sample, the larger the reduction in signal measured at working electrode (508) following removal of immuno-complexes by magnet (514). Conversely, when little or no insulin is present in the sample, little or no reduction in signal occurs at working electrode (508) upon activation of magnet (514). d) Alternatively, the magnet (514′) can be situated at the working electrode in the detection zone (513). e) When activated, said magnet (5141 can now attract the second antibody-magnetic bead complexes to generate electrons at the working electrode, where the substrate (704) is present, in an amount proportional to the amount of insulin present in the sample. Such an assay may include a step to eliminate the non-bound enzyme label in order to increase the sensitivity and accuracy. This can be done through capillary forces for generating flow of the blood sample through the reaction zone and/or for eliminating non-bound complexes, or can be done by additionally adding an absorption pad or a capillary flow inducing means (e.g. the test strip itself) at the end of the detection zone (513) or capillary tract (503). Optionally, a reservoir with fluid, connected to said reaction zone (512) can be present to allow a better washing step.

### Type-1-Diabetes Mellitus

Type-1-diabetes mellitus (T1DM), is typically characterized by recurrent or persistent hyperglycemia, and is diagnosed by demonstrating any one of the following:
Fasting plasma glucose level at or above 7.0 mmol/L (126 mg/dL),
Plasma glucose at or above 11.1 mmol/L (200 mg/dL) two hours after a 75 g oral glucose load as in a glucose tolerance test,
Symptoms of hyperglycemia and casual plasma glucose at or above 11.1 mmol/L (200 mg/dL).
   (cf. World Health Organisation: Department of Noncommunicable Disease Surveillance (1999). “Definition, Diagnosis and Classification of Diabetes Mellitus and its Complications”)

In addition, the appearance of diabetes-related autoantibodies has been shown to be able to predict the appearance of diabetes mellitus type 1 before hyperglycemia arises: islet cell autoantibodies, insulin autoantibodies, autoantibodies targeting the 65 kDa isoform of glutamic acid decarboxylase (GAD) and autoantibodies targeting the phosphatase-related IA-2 molecule are known to be important.

Although T1DM is not actually preventable, promising therapies are slowly emerging, and it has been suggested that, in the future, T1DM may be prevented at the latent autoimmune stage, probably by a combination therapy of several methods (Bluestone et al., 2010, Nature 464 (7293): 1293). Early detection of T1DM is of course of great importance herein and the present application provides an easy tool that will allow screening of risk populations. Cyclosporine A, an immunosuppressive agent, can be used to halt destruction of beta-cells. Also anti-CD3 antibodies, including teplizumab and otelixizumab, have evidence of preserving insulin production (as evidenced by sustained C-peptide production) in newly diagnosed T1DM patients. An anti-CD20 antibody, rituximab, inhibits B-cells and has been shown to provoke C-peptide responses three months after diagnosis of T1DM, but long-term effects of this have not yet been reported. Furthermore, injections with a vaccine containing GAD65, an autoantigen involved in T1DM, has delayed the destruction of beta-cells in clinical trials when treated within six months of diagnosis (Bluestone et al., 2010, Nature 464 (7293): 1293).

T1DM is usually treated with insulin replacement therapy. This can be done using subcutaneous injection of insulin or with an insulin pump, along with attention to dietary management (especially carbohydrates), and monitoring of blood glucose levels using glucose meters which can be simply operated by the patient himself. Also the insulin injections are usually performed by the patients themselves. Untreated T1DM commonly leads to coma, often from diabetic ketoacidosis, which can be fatal. In some cases pancreas transplantation or islet (beta) cell grafting is used as a form of treatment to restore proper glucose regulation. This is however a very severe intervention, both at the level of surgery and the accompanying immunosuppression in order to prevent rejection of the transplanted tissue. Long-term monitoring and follow up of successful transplantation is required and the present invention also provides the tools for aiding in such monitoring by measuring both glucose and insulin levels in blood at the same time, reflecting beta-cell activity. A restored activity (to normal reference levels) or a maintained or reduced activity after transplantation can be followed by measuring both glucose and insulin in blood. Beta cells can be derived from a pancreatic transplant or from stem cells.

T1DM can have a long disease development and can start long before clinical signs become apparent. Several stages are defined: 1) No-T1DM, with normal beta-cell function and mass, 2) pre-onset T1DM, with emerging auto-antibody titers towards beta-cells, due to e.g. inflammatory reactions; 3) early-onset T1DM with initial beta-cell destruction; first clinical signs such as disturbed Oral Glucose Tolerance Test; 4) newly-onset T1DM, T1DM being treated with insulin, resulting in the so-called honeymoon period of amelioration of blood glucose homeostasis due to recovery of remaining beta cells; 5) after said honeymoon period, the beta-cell destruction is progressively continued and patients become totally dependent on exogenous insulin administration.

The honeymoon period for patients with T1DM is the period after the disease is diagnosed and insulin treatment is started. During this period some of the insulin-producing beta-cells have not been destroyed. The insulin treatment will in many cases allow the beta-cells to recover and produce some amount of insulin. As a result the doses of injected insulin can be decreased and blood sugar control is improved. The honeymoon period does not occur in all patients and normally only last for a couple of months to a year.

T1DM hence is an autoimmune disorder, which can be triggered by both genetic predisposition and numerous environmental factors such as: viral or bacterial infection or other allergens in e.g. cow milk or wheat or use of chemicals and drugs. All this causes an initial inflammation in the pancreas. Due to this, part of the beta-cells can be destroyed, which will trigger them to proliferate, generating even more antigens, which can then cause an auto-immune reaction destroying even more beta-cells. This chain reaction cannot be followed or predicted by any means at the moment, but it is established that clinical manifestation of T1DM reflects the consequence of an underlying, sustained autoimmune process. For instance, auto-antibodies against islet antigens are detected before the clinical onset of T1DM. This suggests that a sequence of inciting events precedes the hyperglycemia for at least months, but most likely several years. This wide gap between initiation and detection of ongoing diabetogenic events poses a cardinal problem in the search for causative environmental triggers (cf. Van Belle et al., 2011 for review). Using the device and method according to the invention will allow the follow up or monitoring of patients with a high risk (e.g. predisposed) of developing T1DM. The test can e.g. be performed daily, weekly or monthly, based e.g. on the clinical history and diet of the patient. The device according to the present invention will yield a value of insulin resistance or sensitivity, or remaining beta-cell activity or function in the subject. This information can also be used to prescribe, monitor or fine-tune the therapeutic use of immunosuppressants, immunomodulators, antibody therapies, vaccines or desensibilisation cures, that can slow down or halt the destruction of the beta-cells in said subject.

The invention thus provides for the use of the test device according to the invention, for monitoring the beta-cell activity in pre-diabetes subjects that have a certain risk of becoming T1DM and for determining an appropriate immunotherapy, or to monitor or fine-tune said therapy by e.g. immunosuppressants, immunomodulators, anti-body therapies, vaccines or desensibilisation cures. At different stages of the destruction process, reflected by different degrees of loss of beta cell function and different degrees of decrease in insulin blood levels, different interventions may be needed for obtaining the best results. The dose of the treatment can be adjusted more appropriately using the real-time glucose and insulin level measurement of the invention. The test device according to the invention may thus help in lowering the effective dose of immunomodulatory or immunosuppressive therapy. Alternatively, the device can be used to trigger the choice of a more appropriate intervention based on worsening of the insulin resistance or worsening of beta-call function justifying a more aggressive substance or dose.

Once T1DM is fully established, the remaining beta-cell activity of the patients is often non-existing or too low to regulate the blood-sugar homeostasis and administration of exogenous insulin is needed. The device and method according to the present invention can be used to determine the actual need for insulin at the time of blood glucose monitoring and calculation of the insulin bolus dose, e.g. before every meal. Typically, T1DM patients will have to administer a certain amount of long-acting insulin to have a base line level of insulin in their system and a bolus amount of short-acting insulin just before each meal. The base level is given by a long acting Insulin which is administered once per day. The bolus short acting insulin needs to be given before each meal, usually 3 times a day. This bolus needs to be injected before every meal, in order to be able to properly take up the sugars released from the meal. Nowadays, complicated schemes exist that allow T1DM subjects to calculate the bolus dose of insulin needed before a meal, based on their current glucose level, the amount of carbohydrates in their anticipated meal and two factors:
1. the insulin/carb ratio, and
2. the glucose correction factor.

Based on this scheme, the subject calculates the amount of (short acting) insulin needed for the bolus injection (cf. information on https://dpg-storage.s3.amazonaws.com/dce/resources/Insulinto_Carb_Slick.pdf).

The “insulin to carb ratio” is used to know the amount of insulin needed to absorb the carbohydrates in the next meal. It is the amount of insulin needed to absorb 15 grams of carbohydrates in the meal. If the insulin to carb ratio is 1.5; then the patient needs 1.5 units of insulin for each 15 grams of carbohydrates in his next meal. In case that patient was to eat 60 grams of carbohydrates, then he would need 60 grams/15 grams X 1.5 units=6 Units of insulin. This insulin/carb ratio is given to the patient by the doctor at the time of diagnosis of his diabetes and is changed when the doctor sees the need for it, on future consultations. In reality however, this value changes from individual to individual and day to day, depending on the person's insulin resistance. This insulin to carb ratio changes over time and between individuals.

The “glucose correction factor” is the amount of insulin needed to bring down the measured glucose level to the target range. For example when a patient has a glucose level of 250 mg/dL, and his target upper-limit is 150 mg/dL, he needs to lower his blood glucose level with 100 mg/dL. When the glucose correction factor is 30, then the patient needs to inject 100/30=3.3 Units insulin to lower his glucose to the target range.

To know the total bolus amount, these 3.3 Units of insulin need to be added to the amount of insulin needed to digest the meal, as explained above for the calculation of the insulin to carb ratio.

The glucose correction factor is nowadays set by the healthcare consultant but is in fact a measure for insulin resistance. It is crudely calculated based on the patient's Total Daily Dose of insulin (long acting+all the boluses) and a number from 1800 to 2200 (depending on the kind of insulin the patients uses). The glucose correction dose in a patient who uses 20 units would be 1800/20=90 to 2200/20=110. The glucose correction dose is the number of mg/L that the blood glucose will drop for every unit of insulin injected. For a patient on 20 units of insulin/day:1800/20U=a 90 mg/dL drop per unit of insulin (Humalog). Whether the doctor would use 1800, 2200 or any number there between to determine the glucose correction factor depends on the patient's insulin sensitivity and the kind of insulin that is used.

The current bolus calculation schemes use the same insulin/carb ratio and the glucose correction factor for every meal and every day during several months. It usually requires severe and very clearly detectable higher or lower glucose values for the doctor to identify glucose deviation patterns and adjust the insulin/carb ratio and glucose correction factor in the formula. The insulin resistance varies from person to person, from day to day and from hour to hour. For example, stress (high levels of cortisol, adrenalin and noradrenalin) increase insulin resistance causing the insulin to be less effective. Fever, also increases the insulin resistance temporarily. Many other factors have a lowering effect in Insulin resistance: alcohol consumption, a hypoglycemic episode during the night, a bout of growth hormone (typical in puberty and adolescence), an exercise session.

The large variability of the insulin resistance by many factors makes it very difficult to identify the correct glucose correction factor and insulin to carb ration by analysing glucose data only.

The present invention avoids long term glucose deregulation by calculating the insulin sensitivity on-the-spot and at the moment (real-time), by measuring both the glucose and insulin level in the blood sample prior to taking the meal. This is a real-time reflection of the insulin resistance in the subject, which enables a much more precise calculation of the insulin to carb ratio and of the glucose correction factor. This results in a more correct dosage calculation of the insulin needed for a bolus injection. The present invention hence provides means for calculating a “real-time insulin sensitivity adapted insulin-to-carb ratio” and a “real-time adapted insulin sensitivity glucose correction factor”. The “real-time insulin sensitivity adapted insulin-to-carb ratio” is a corrected insulin-to-carb ratio, based on the difference between the presupposed insulin-sensitivity (the IS calculated by a practitioner at e.g. the start of the treatment or monitoring) and the rea-time insulin-sensitivity (IS calculated based on actual insulin and glucose levels in the subject using the device and method according to the invention). The ratio of both IS values results in a correction value, which is used to calculate the more accurate “real-time insulin sensitivity adapted insulin-to-carb ratio”. The present invention hence provides a test device and method that uses a “real-time insulin sensitivity adapted insulin-to-carb ratio” and a “real-time adapted insulin sensitivity glucose correction factor” to calculate a more appropriate bolus quantity of insulin to be administered to a subject in need thereof.

The “real-time insulin sensitivity adapted insulin-to-carb ratio” is a corrected insulin-to-carb ratio, based on the difference between the presupposed insulin-sensitivity (the IS calculated by a practitioner at e.g. the start of the treatment or monitoring) and the rea-time insulin-sensitivity (IS calculated based on actual insulin and glucose levels in the subject using the device and method according to the invention). The ratio of both IS values results in a correction value, which is used to calculate the more accurate “real-time insulin sensitivity adapted insulin-to-carb ratio”.

Similarly, the “real-time adapted insulin sensitivity glucose correction factor” is a corrected glucose correction factor, based on the difference between the presupposed insulin-sensitivity (the IS calculated by a practitioner at e.g. the start of the treatment or monitoring) and the real-time insulin-sensitivity (IS calculated based on actual insulin and glucose levels in the subject using the device and method according to the invention). The ratio of both IS values results in a correction value, which is used to calculate the more accurate “real-time adapted insulin sensitivity glucose correction factor”. Similarly, the “real-time adapted insulin sensitivity glucose correction factor” is a corrected glucose correction factor, based on the difference between the presupposed insulin-sensitivity (the IS calculated by a practitioner at e.g. the start of the treatment or monitoring) and the real-time insulin-sensitivity (IS calculated based on actual insulin and glucose levels in the subject using the device and method according to the invention). The ratio of both IS values results in a correction value, which is used to calculate the more accurate “real-time adapted insulin sensitivity glucose correction factor”.

It is important to notice that the real real-time insulin resistance adaptation of the insulin-to-carb ratio and the glucose correction factor might require a calibration or fractional factor. However, the essence of the invention is to include the actually measured insulin resistance into the formula to calculate the bolus amount of insulin.

Finally, the “Insulin On Board”, i.e. the insulin remaining in the sub-cutis from the previous injection will also play a role in calculating the amount of bolus insulin. This amount of insulin, called the Insulin On Board, is subtracted from the previously calculated amount of insulin. Usually, the user will estimate the remaining insulin (the Insulin On Board) by determining how long ago the previous injection of insulin was introduced into the sub-cutis. This way of estimating Insulin On Board (IOB), based on the time delay since the last injection, is rather crude. Various factors will influence insulin uptake from the subcutaneous injection site into the bloodstream (injection in the sub-cutis of the abdomen versus arm, cicatrised tissue versus vital tissue, vasodilatation due to ambient temperature versus vasoconstriction, stressful moment versus a relaxed moment). Therefore the residual IOB determined solely based on the amount of time elapsed since last injection is often inaccurate. The present invention provides means for more accurate determination of IOB, based on a measurement if blood insulin and blood glucose. Based on the time and the amount of insulin injected at previous event, coupled with the measured level of insulin concentration determined immediately prior to the next injection, a more accurate determination of residual IOB leads to greater control over quantity of insulin required to be injected thereby resulting in reduced number of hypoglycaemic events, particularly during sleep.

### Monitoring Beta-Cell Replacement Therapy

In a further aspect, the device and method of the present invention can be used for beta cell transplantation or pancreas transplantation surveillance. Measuring the level of glucose and insulin or C-peptide in a blood sample of the subject allows to calculate the beta-cell function (e.g. by using HOMA-B %) reflects the total beta cell activity and hence an increased presence of insulin after transplantation indicates that the grafted beta cells are indeed active and that hence the transplantation was successful. Monitoring the level of glucose and insulin in the blood of the subject over time thus will enable the surveillance of the survival rate of the transplanted beta cells. Such a beta cell function is easily done by calculating the Oral Disposition Index. After an oral load of 75 gr of glucose (in a drink), the blood is sampled at 0 and 30 minutes. In those samples the glucose and Insulin or C-peptide is measured. The Oral Disposition Index is the product of the change in insulin divided by change in glucose (ΔI/ΔG)×insulin sensitivity). In addition, the immune-suppression treatment can be fine-tuned based on the above results of the monitoring or surveillance process measuring both glucose and insulin simultaneously in a blood sample of the subject. One can envisage increased immunosuppression therapy, in case of a change in beta cell activity (reflected by changing glucose and insulin levels in the blood sample), or maintained or decreased immunosuppression therapy in case the grafting process seems promising, i.e. when the beta cells are actively producing insulin and the blood glucose levels are in a steady state, or are comparable to those of a healthy subject. Transplantation rarely substitutes the total need for insulin. Typically the patient will require some exogenous insulin injections to supplement the insufficient production of the grafted cells. In such case, the measurement of the C-peptide, reflecting the endogenous production and/or the measurement of the recombinant injected insulin may help to monitor the grafted cells.

### Type-2-Diabetes Mellitus and Insulin Resistance

Type 2 diabetes mellitus (T2DM) is mostly caused by insulin resistance and eventually result in beta-cell exhaustion, leading to insufficient beta-cell activity. T2DM is a condition in which body cells initially fail to use insulin correctly, subsequently beta-cell function becomes severely impaired, and ultimately there becomes an absolute insulin deficiency, requiring external administration of insulin. T2DM is also known as non-insulin-dependent diabetes mellitus (NIDDM) or adult-onset diabetes. Insulin resistance is the defective responsiveness of body tissues to insulin and is believed to involve the insulin receptors and intracellular glucose transporters although the specific defects are yet unknown. In the early stage of T2DM, the predominant abnormality is reduced insulin sensitivity (=increase in Insulin Resistance). At this stage hyperglycemia can be reversed by a variety of measures and medications known in the art. T2DM develops from insulin resistance, meaning that the normally secreted dose of insulin is no longer sufficient to control blood glucose levels. In a reaction to this process, beta-cells are forced to produce more insulin, or are triggered to proliferate and/or granulate, producing more insulin. This overproduction of insulin or over activity of beta-cells can then lead to beta-cell exhaustion, leading to reduction of the functional beta-cell population. This process can now be more accurately followed using the method and device of the present invention, which allows for the simultaneous detection of both blood glucose and blood insulin levels. From these levels the insulin resistance can be calculated using known formulas called HOMA1-IR, HOMA2-IR or other commonly used formula as described in Table 1 (above). Insulin resistance syndrome or simply metabolic syndrome or metabolic syndrome X is one of the pathophysiological conditions that cause or underlie T2DM and can be linked to both genetic predisposition and many environmental factors such as diet, stress, overweight, aging, certain infections, coronary heart disease etc. In the presence of small deterioration or changes in glucose levels the situation may also be referred to as pre-diabetes.

In an additional aspect, the present invention thus allows the identification of patients with a degree of insulin resistance or enables the assessment of the degree of said insulin resistance. In principle, when more insulin synthesis is needed to preserve a certain glycemic control, the patient might be called to have “insulin resistance”. In patients with Insulin resistance, blood glucose values can stay within normal ranges for many years. Only when the beta-cells cannot cope with the increased insulin demand, glucose levels start to rise. First after the meals (=pre-diabetes) and later also the fasting values in the morning. The elevated glucose values in the morning are diagnostic for diabetes. Treating insulin resistance allows to preserve beta-cell function longer (years), effectively preventing the evolution towards T2DM. Measuring the insulin resistance in real-time by assessing both the level of glucose and insulin in the blood of a subject is hence a huge advantage of the device and method of the present invention. The device and method of the invention improve the practicality and ease of use of calculating the insulin resistance automatically at home or at the practitioner's (point of care test). The well-known HOMA formulas (HOMA1-IR, HOMA2-IR and HOMA-B %) can be incorporated in the device and method of the present invention, which will yield an immediate insulin resistance value based on the actual blood glucose and insulin level measured. Measuring simultaneously glucose and insulin levels in a blood sample, in order to detect or predict the onset of insulin resistance clearly is advantageous over all the known techniques.

The device and method of the present invention can be used to automatically establish the level of insulin-resistance instantaneously, at every desired point in time, without the need to send a blood sample to the laboratory. Exercise for instance, changes insulin resistance overnight. The T2DM patients on an exercise regimen can see the effects of his effort on his insulin resistance the next day. Seen these regimens require exercising 3-5 times a week the only practical way to motivate the patient is to have these measurements available at home in real time.

In overweight (obese) subjects and patients with metabolic syndrome, the device and method of the present invention can be used to monitor the insulin resistance and schedule a treatment in order to postpone the evolution towards T2DM.

The device and method of the present invention can be used to establish exercise and training schemes and diets for overweight subjects or subjects with metabolic syndrome. It will help motivating the subjects, because they can immediately see the effect of e.g. training session or exercise on their insulin resistance value.

The device and method of the present invention can also be used to select those patients with overweight that would benefit from a change in lifestyle e.g. a diet change or the use of certain exercise program.

In early T2DM subjects, the device and method of the present invention can also be used to monitoring beta-cell function for fine-tuning glucose control.

The device and method of the present invention can also be used for pregnancy monitoring of pregnancy-related diabetes.

The above aspects and embodiments are further supported by the following non-limiting examples.

### EXAMPLES

### Example 1

### Examples of Electrochemical Blood Glucose and Insulin Detection Test Strips

### a) Blood-Glucose Detection Strip:

Screen printed working and reference electrodes are prepared on a disposable test strip which can receive a drop of blood. To the working electrode, an amount of glucose-oxidase is attached, in combination with an amount of electron-transfer mediator. The glucose in the blood sample brought onto the test strip is oxidized by the glucose-oxidase present on the working electrode, thereby releasing a proportional amount of electrons, transferred by the mediator to the reference electrode. The current measured between both electrodes is proportional to the amount of glucose in the blood sample.

### b) Blood-Insulin Detection Strip:

In this example, insulin detection based on an electrochemical immunoassay detection system is described, wherein an insulin-specific antibody is labeled with a charged molecule or particle. Said antibody is present in the reaction zone of the test device and is brought into contact with the blood sample through capillary forces. Upon binding of the insulin with the labeled-antibody, said complexes are trapped by a second insulin-specific antibody, linked to a magnetic particle, which is attracted to the reaction zone by magnetism.

The analyte detection zone comprises a set of electrodes, capable of inducing and receiving an electric charge and/or current between them. Two opposite charged electrodes form an electrode couple and optionally a reference electrode in the middle of said couple is present for ease of detection of the current produced.

The fixed antibody-insulin-antibody-charged-label complex is then drawn to an opposite charged electrode by inducing an electric charge between both electrodes.

The antibody-analyte complexes are now attracted to the opposite charged electrode (e.g. positive charged particles will be attracted to the negative pole of the electrode couple).

The polarity of the electrodes is then reversed, thereby releasing the complexes and moving them to the opposite electrode. At the moment of the release, the current is measured between both electrodes. The measured total current received at the second electrode or at the reference electrode is proportional to the amount of complex that was displaced from the first electrode, since it will be the sum of the current induced and that caused by the complexes attracted thereto.

### c) Combined Insulin-Glucose Detection Device

In this example, a device for detecting both the glucose and insulin level in a whole blood sample of a subject is described comprising a disposable test strip which can receive a drop of blood. Said strip comprises a) a sample receiving part; and b) an analyte reaction zone comprising: b1) a first electrochemical or optical sensor for detecting the blood glucose level in said sample, and b2) a second electrochemical or optical sensor for detecting the blood insulin level in said sample. The sample is directed to the different zones through multiple microfluidic channels on the strip. The device further comprises c) a controlling device that can control the operation of the device and analyse the data obtained from the biosensor systems; and d) a user interface, displaying the data to the user.

Said first sensor b1) for detecting glucose comprises a screen printed working and counter/reference electrode on the disposable test strip. To the working electrode, an amount of oxidoreductase enzyme, for example glucose oxidase or glucose dehydrogenase is attached, in combination with an amount of electron-transfer mediator. The glucose in the blood sample brought onto the test strip is oxidized by the oxidoreductase present on the working electrode, thereby releasing a proportional amount of electrons, which are transferred by the mediator to the counter/reference electrode. The current measured between the working and counter/reference electrodes is indicative to the amount of glucose in the blood sample. FIG. 5 exemplifies this process.

Said second sensor b2) for detecting insulin is an electrochemical sensor, measuring a change in charge or current due to enzymatic reaction with a substrate upon binding of insulin, more particularly an enzyme-linked immunomagnetic electrochemical assay. Said assay comprises: an electron-releasing enzyme system coupled to an insulin-specific antibody and secondary insulin-specific antibodies, linked to magnetic particles.

Upon contact with its substrate, an electron is formed by said enzyme and the current obtained through said enzymatic activity is measured. The electron-transfer mediated by this enzyme system is then registered on a screen printed working (and counter/reference) electrode on the disposable test strip. FIG. 6 exemplifies this process.

In order to avoid any washing steps, magnetic particles, linked to the second anti-insulin antibodies, are used to withdraw any insulin-bound enzyme complexes (complexed through a first anti-insulin antibody). The subsequent reduction in current signal generated at the working electrode versus the initial current signal prior to withdrawal of magnetic particle/insulin complexes is proportional to the amount of insulin present in the sample.

FIG. 6 exemplifies this process: a) The sample comprising insulin (703), is directed towards the sample reaction zone (512). b) In the reaction zone, the insulin is bound by two antibodies: a first antibody, complexed with the enzyme label (701), and a second antibody, complexed with a magnetic particle (702), both present in the reaction zone. The enzyme label (701) will metabolise its substrate (704) present in the detection zone in the presence of an electron mediator, thereby releasing electrons, which are detected by the working electrode (508), placed in the detection zone. c) Outside the detection zone (513), e.g. in the reaction zone (512), a magnet (514) is placed, which upon activation (514*), will draw away all magnetic bead-second antibody complexes from the detection zone. When insulin is present, antibody-magnetic particle-insulin will form. Such complexes are susceptible to a localised magnetic field, and as such will be attracted to the activated magnet (514*) along with any of the first antibody-enzyme complex that has formed “sandwich” complexes with the target, insulin. Removal of first antibody-enzyme complexes from the reaction zone (512) leads to a reduction in reaction between enzyme label and substrate at the working electrode (508). This reduces the amount of electrons produced at the site of the working electrode (508) and detection zone (513). Both signals 503a and 503a* can be detected by a reader. The difference in number of electrons formed at the working electrode before and after activation of the magnet is proportional to the amount of insulin in the sample. The greater the amount or concentration of insulin present in the sample, the larger the reduction in signal measured at working electrode (508) following removal of immuno-complexes by magnet (514). Conversely, when little or no insulin is present in the sample, little or no reduction in signal occurs at working electrode (508) upon activation of magnet (514).

### d) Actual Test Measurement of Insulin and Glucose Level in a Small Blood Sample:

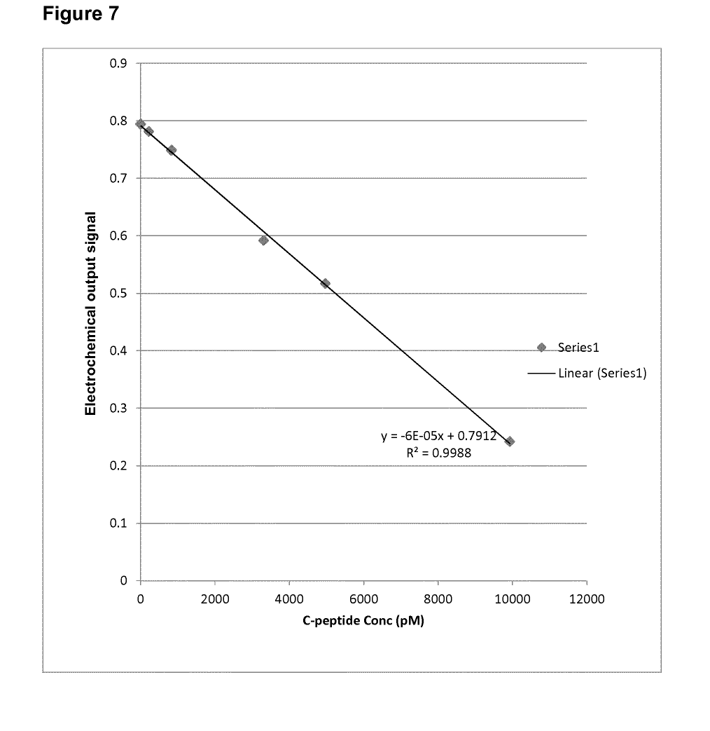

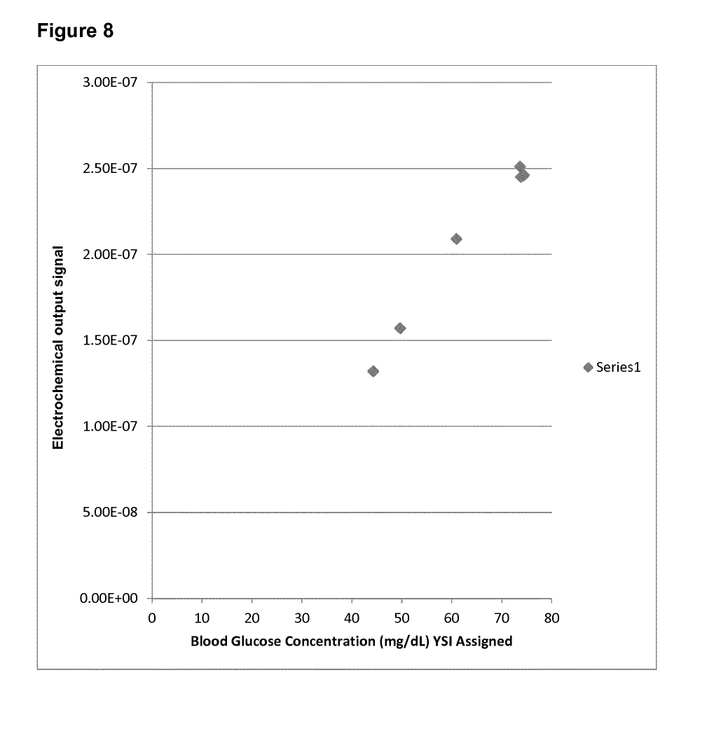

For this initial test, a small volume of whole blood (5 microliter) was spiked with a known concentration of C-peptide (part of insulin) and said samples (6 in total) were introduced at the sample receiving part (501) of the device as outlined in point c) above. Subsequently, the reagents were left to incubate for about 2 to 3 minutes and the concentration of insulin (FIG. 7) and glucose (FIG. 8) was measured in the reader using the steps as outlined in point c) above (sensor b2 and b1 respectively). The blood samples were taken from healthy subjects. As can be seen from FIG. 7, insulin concentrations from 0 to 10.000 pM could be measured in a 5 microliter blood sample. The amount of insulin (C-peptide) was calculated based on the difference of electrochemical current measured after the magnetic field is activated and withdraws bound magnetic-bead-antibody-insulin-antibody-label complexes from the reaction zone and the total electrochemical current measured before said magnetic field is activated and all label is still present. The blood glucose concentration was calculated based on the electrochemical signal obtained using the methodology outlined in step c) above (sensor b1).

This example provides the proof of concept that quantitative electrochemical measurement of insulin (or C-peptide) and glucose can be done in a small volume of whole blood (5 microliter).

### Example 2

### Examples of Optical Blood Glucose and Insulin Detection Test Strips

Colorimetric Blood Glucose Test:

As an example, the test strip uses a colorimetric reaction following the formation of hydrogen peroxide by the glucose oxidase enzyme oxidizing glucose present in the blood. The test strip further encompasses a benzidine derivative, which is oxidized to form a blue-colour polymer by the hydrogen peroxide formed in the oxidation reaction. The amount of colored complex formed on the test strip is measured by trans-illuminating the test strip and detecting the amount of light transferred through the strip. The less light detected, the more complex formed and the higher the glucose concentration in the blood sample.

### Colorimetric Blood Insulin Test:

In this example, the detection of insulin in the blood sample is based on pure immunological techniques, employing ELISA technology on a micro-scale in the reaction zone of the device, i.e. the microporous test strip, providing the needed capillary flow to drive the analyte over the reaction zone. Arriving at the reaction zone, the insulin is bound by insulin-specific antibodies. These insulin-antibody complexes are next trapped by second insulin-specific antibodies that are fixed to the reagent zone. The fluid present in the blood sample, in combination with the capillary forces of the test strip, acts as a “washing” step of unbound and hence unwanted contaminants. The labeled antibody-analyte-antibody complex can then be detected at the reaction zone by optics detecting the label on the first antibody. The amount of labeled complexes will determine the amount of analyte present in the sample.

The test strips and measurement technologies of examples 1 and 2 can of course be combined resulting e.g. in an optical detection of insulin and a colorimetric detection of blood glucose or vice versa. The device can of course employ a single measurement technology, e.g. both insulin and glucose are measured using electrochemical techniques or both glucose and insulin are measured using optical techniques.

### Example 3

### Comparison of Calculation of Insulin-Resistance in a Type-1 Diabetes Mellitus Patient and the Use of it in Dosing the Insulin Bolus, Using Standard Formulas or Using the Device and Method According to the Invention

One of the ways to calculate Insulin resistance is by using the Homeostasis Model Assessment of Insulin Resistance (HOMA-IR). The method uses a fasting blood glucose and fasting blood insulin level. The formula is: fasting glucose level X fasting insulin level/22.5. The formula has been widely used in population studies of normal, overweight and T2DM people but may also be beneficial in T1DM. However, since the biofeedback loop between insulin production and glucose is absent in type I diabetes, we may not use HOMA-IR in its original meaning. The product of insulin and glucose may however still give an idea of the Insulin resistance.

The type 1 diabetes mellitus patient has to inject a bolus of insulin prior to each meal. The bolus aims to 1) restore an elevated or abnormal low glucose level prior to the meal and 2) absorb the carbohydrates coming with the meal. These are 4 steps for calculating the dose as they are instructed to a T1DM patient:
1. Step 1: Calculate the insulin dose for the food:
   a. Add up the grams of carbohydrate in the food that you will eat.
   b. Divide the total grams of carb by your insulin-to-carb ratio.
   Total Grams of Carbohydrates to be Eaten
   Insulin-to-Carb Ratio

Example: A subject plans to eat 45 grams of carbohydrates and his insulin-to-carb ratio is 1 unit for every 15 grams of carbohydrates eaten. To figure out how much insulin to administer, divide 45 by 15=3 units of insulin
2. Step 2: How to use the glucose correction factor to reach the target blood glucose level
   a. Subtract the target blood glucose level from the currently measured blood glucose level.
   b. Divide the obtained difference in a. by the glucose correction factor.
   Current blood glucose—Target blood glucose
   Glucose Correction Factor

Example: A subject checks his pre-meal blood glucose and it is 190 mg/dL, while the blood glucose target of the subject is 120 mg/dL. The glucose correction factor is 35, so: (190 mg/dL-120 mg/dl)/35=2 units of insulin that will bring the subject's blood glucose level down from 190 to 120 mg/dL.
3. Step 3: Add the insulin needed for digesting the carbohydrates up with the insulin needed to bring down the blood glucose, to calculate the total bolus dose of insulin needed.

Example: from step 1 and 2:3 Units for carbohydrates+2 units for blood-glucose correction=5 units.

Nowadays, the insulin-to-carb ratio and the correction factor are the same for the 3 boluses that day and all the days until the next consultation session when the doctor may decide to change them.
4. Step 4: subtract the Insulin On Board. Based on tables the patient can estimate how much insulin is left to act since his last injection.

Insulin Left At 1, 2, 3, and 4 Hours After A Dose Of Humalog Or Novolog

| | | Units Left To Work After: | | | | |
|---|---|---|---|---|---|---|
| Dose Given | | 1 Hr | 2 Hr | 3 Hr | 4 Hr | 5 Hr |
| 1 | unit | 0.80 u | 0.60 u | 0.40 u | 0.20 u | 0 |
| 2 | units | 1.60 u | 1.20 u | 0.80 u | 0.40 u | 0 |
| 3 | units | 2.40 u | 1.80 u | 1.20 u | 0.60 u | 0 |
| 4 | units | 3.20 u | 2.40 u | 1.60 u | 0.80 u | 0 |
| 5 | units | 4.00 u | 3.00 u | 2.00 u | 1.00 u | 0 |
| 6 | units | 4.80 u | 3.60 u | 2.40 u | 1.20 u | 0 |
| 7 | units | 5.60 u | 4.20 u | 2.80 u | 1.40 u | 0 |
| 8 | units | 6.40 u | 4.80 u | 3.20 u | 1.60 u | 0 |
| 9 | units | 7.20 u | 5.40 u | 3.60 u | 1.80 u | 0 |
| 10 | units | 8.00 u | 6.00 u | 4.00 u | 2.00 u | 0 |

Example: When a user injected 6 units about 4 hours ago, there will be 1.2 units of insulin left. He needs to subtract 1.2 unit from step 3: 5 units-1.2 unit=3.8 units.

Because the present invention measures the insulin at substantially the same time that glucose is measured, the invention allows calculation the real-time insulin sensitivity at the moment that insulin needs to be injected.

The bolus or basal insulin level can hence be adapted to the real-time insulin sensitivity. There are 3 ways of achieving this:
1. Adapting the insulin-to-carb ratio to the real-time adapted insulin resistance. The doctor e.g. established the insulin-to-carb ratio at a certain moment of insulin resistance of X (calculated by HOMA1-IR or similar formula), while the real-time insulin resistance established by the present invention (also by HOMA1-IR or similar formula, but based on real-time values of both glucose and insulin) is Y. Using the ratio of these two IR values, the formula becomes:
   (Total grams of carbohydrates to be eaten)multiplied by Y/X
   Insulin-to-Carb ratio

Example: At the time of establishing the ration the HOMA1-IR was 1.5. Now the HOMA1-IR is 3. So at this moment, in this patient, for this bolus we will have to double the amount of insulin to absorb the carbohydrates in the food. In the same example as above the amount of insulin becomes: 45/15 times 3/1.5=6 Units
2. One can adapt the glucose correction factor in a similar fashion. Assuming that the glucose correction factor was determined when the patient had an insulin resistance (by HOMA1-IR or similar formula) of X and has now, a real-time insulin resistance of Y, then the glucose correction factor can be corrected by multiplying it by Y/X. The formula then becomes:
   (Current blood glucose−Target blood glucose) multiplied by Y/X
   Correction factor

Example: At the time of establishing the glucose correction factor the HOMA1-IR was 1.5. Now the HOMA1-IR is 3. So at this moment, in this patient, for this bolus we will have to double the amount of insulin to bring down the glucose to the target range. In the same example as above the amount of insulin becomes: (190−120 mg/dL)/35 times 3/1.5=4 units.

The subject can now add up the two real time insulin resistance adapted amounts of insulin to calculate the total bolus to be injected: 4 units+6 units=10 units. It is important to notice that the real-time insulin resistance adaptation of the insulin-to-carb ratio and the glucose correction factor might require a calibration or fractional factor. However, the essence of the invention is to include the actually measured insulin resistance into the formula to calculate the bolus amount of insulin. A potentially more straight forward way of adapting the bolus amount to the real time insulin resistance is simply taking the traditionally calculated dose and multiplying it with the real time insulin resistance measure (with or without a fractional or constant factor), measured by the system.

### Example 4

### Use of Insulin Resistance in Adjusting the Basal Insulin Requirement

In Patients with a Single Basal Insulin Injection:

The basal requirement of insulin is filled in with a once a day injection of long (>24 hours) acting insulin. This dose is driven, among other things, by the insulin resistance of the patient. The amount basal insulin can be adapted to the real time insulin resistance by using the measured HOMA-IR (or similar formula) as a correction factor. The new rate becomes then:

Basal rate as established times HOMAR-IR real time/HOMA-IR established=real time insulin resistance adapted basal rate.

In Patients with an Insulin Pump:

The basal rate of a patient varies from work day to weekend day, days with exercise versus days without exercise, sick days, certain days during the menstrual cycle etc. The insulin resistance changes throughout the day. A clear example is the basal rate profile that insulin pump patients use that varies from hour to hour. They program different rates of a continuous drip of insulin from a pump for every hour of the day. Typically they need more insulin in the morning when their cortisol levels and free fatty acid levels are high. These two substances are known to increase insulin resistance. Adolescents and children will experience a growth hormone peak in the late afternoon and also require a higher amount of insulin to maintain normal glucose levels. Rather than programming by trial and error, we can adapt the basal rate to the real insulin resistance by measuring it and feeding this back to the pump system.

The new basal rate profile could for example be the normal basal rate profile multiplied by the ratio of the real HOMA-IR (or similar formula) over the averaged HOMA-IR (or similar formula).

### People Taking Insulin and “Sick Days”

A patient with an infection and fever has increased stress hormones and cortisol. The Insulin resistance increases as a consequence. The basal rate is markedly increased when the patient is having a fever. Sick days are currently dealt with by adding 10-20% of the normal total daily insulin requirement as an extra injection of fast acting insulin every 4 hours till normalization of glucose levels. This invention would allow to fine tune this regimen by also taking into account what the possible effect will be of the administered insulin on the glucose levels.

### Example 5

### Use of Insulin-Resistance and Calculation of Beta-Cell Function in a Type-2 Diabetes Mellitus Patient

T2DM patients are often given an amount of insulin not only to tackle high glucose levels but also in trying to preserve as many beta-cells as possible. Also lifestyle changes such as regular exercise and weight loss improve insulin resistance, reduce the requirement of insulin secretion and consequently preserves beta-cell function. Similar reductions in insulin resistance are seen with medications other than insulin i.e. thiazolidines (pioglitazone, rosiglitazone)

Beta-cells are incredible sensitive glucose sensors, insulin synthesizers and insulin pumps all at the same time. Preserving their function allows fine tuning of the glucose levels. Their function can be measured by measuring glucose and insulin levels in blood and adapting the HOMA formula to HOMA-B % which reflects beta-cell function. HOMA-B %=(20× fasting insulin)/(fasting glucose−3.5) (Published by Dr. David Matthews). This measure allows keeping track of the loss of beta-cell function and thus allowing to step up lifestyle or pharmaceutical intervention to preserve as long as possible the glucose fine tuning capacity.

Especially C-peptide is interesting to use with this formula. T2DM patients are increasingly treated with insulin to reduce the need for endogenous secretion and thus preserving beta-cell function. By measuring C-peptide, rather than insulin (or insulin analogues) the result is not contaminated by the exogenously injected insulin and results in a true measure of beta-cell function.

Other formula's can be used to calculate beta cell function. The Oral Disposition Index is a good example: After an oral load of 75 gr of glucose (in a drink), the blood is sampled at 0 and 30 minutes. In those samples the glucose and Insulin or C-peptide is measured. The Oral Disposition Index is the product of the change in insulin divided by change in glucose (ΔI/ΔG)×insulin sensitivity).

### Example 6

### The Use of Insulin Resistance and Beta-Cell Function in Overweight and Metabolic Syndrome Patients

Just like in T2DM patients, therapy aims at preserving beta-cell function with very similar interventions like medication and lifestyle changes. The benefit of intervening soon and effectively is that these measures prevent the evolution to T2DM. Prolonged survival of sufficient and healthy beta-cells avoids the development of diabetes.

By restoring insulin resistance to normal levels, the beta-cells are relieved from their overdrive situation. This can e.g. be efficiently done with Pioglitazone that reduces the incidence of newly diagnosed T2DM with more than 50% after 3 years. Metformin has a similar effect, albeit with less spectacular results. Both medications come with side effects such as more weight gain, oedema, risk of heart-failure, hypoglycemia. The device and method according to the present invention thus provide an interesting tool to monitor the effects of and if needed fine tune or change such treatment, since the insulin-resistance can now be measured at any time.

Other therapeutics may be(come) available that can restore, improve or delay deterioration of the beta-cell function, which can also be monitored by the device and method of the present invention.

Lifestyle changes are similarly effective but without the medication side effects. Both weight loss and exercise contribute.

Insulin resistance measured in those patients clearly improves upon administration of Pioglitazone, Metformin and lifestyle changes. The HOMA-IR is very sensitive to reflect the effect. In patients who had an exercise session at 65% of their VO2 max clearly showed a decrease of their HOMA-IR the next day. The beneficial effect of exercise was visible in the HOMA-IR values for 48 to 72 hours after the session. While the effect of exercise on HOMA-IR values was visible from the next day, it took much longer to see the effect of weight loss on HOMA-IR or on the scales.

This fast effect on HOMA-IR makes it an excellent motivational parameter in the home setting. In particular since all the elements used to treat metabolic syndrome patients have their effect on this insulin resistance. The device and method according to the present invention thus provide an interesting tool to motivate exercise and weight loss in patients with T2DM, since it can visualize the insulin resistance value almost immediately upon exercising.

1. A device for detecting both the glucose and insulin level in a whole blood sample of a subject comprising:
a) a sample receiving part;
b) an analyte reaction zone comprising
b1) a first electrochemical or optical sensor for detecting the blood glucose level in said sample,
b2) a second electrochemical or optical sensor for detecting the blood insulin level in said sample,
c) a controlling device that can control the operation of the device and analyse the data obtained from the biosensor systems.
d) a user interface, displaying the data to the user.

2. The device according to claim 1, wherein the controller device calculates the insulin-resistance, insulin sensitivity and/or beta-cell function of the subject based on the signals obtained from sensors b1) and b2).

3. The device according to claim 2, wherein said calculation is done using the HOMA1-IR, HOMA2-IR, Gutt index, Avignon Index, Stumvoll Index, Matsuda Index, HOMA B %, or the Oral Disposition Index formula to determine insulin resistance and beta-cell function in a subject.

4. The device according to claim 1, wherein the detection of both the glucose and insulin level is done in a sample volume of less than 1 ml, preferably less than 0.5 ml, more preferably in less than 100 μl, most preferably in less than 5 μl of whole blood.

5. The device according to claim 1, having a sensitivity of 100 pmol/l, preferably of 50 pmol/l, more preferably of 20 pmol/l for insulin and of 20 mmol/l or less for glucose.

6. The device according to claim 1, wherein said first sensor for detecting blood glucose is a glucose-oxidase or dehydrogenase based electrochemical or colorimetric system.

7. The device according to claim 1, wherein said second sensor for detecting insulin is an electrochemical sensor, measuring a change in charge or current due to enzymatic reaction with a substrate upon binding of insulin.

8. The device according to claim 7, wherein said sensor is selected from the group comprising: electrochemical immunoassays, enzyme-activation electrochemical detection systems, enzyme-linked immunomagnetic electrochemical assays, enzyme-activation immunomagnetic electrochemical assays, and piezo-electrical or di-electrical immunoassays.

9. The device according to claim 7, wherein said electrochemical sensor comprises one or more electrodes or electrode couples, connected to a device capable of inducing and measuring a charge or current in either one of said electrodes.

10. The device according to claim 7, wherein said electrodes are made of an electrically conductive material preferably selected from the group comprising: carbon, gold, platinum, silver, silver chloride, rhodium, iridium, ruthenium, palladium, osmium, copper, and mixtures thereof.

11. The device according to claim 7, wherein said electrodes are porous electrodes, magnetic electrodes, or carbon nanotubes.

12. The device according to claim 1, wherein said second sensor for detecting insulin is an optical sensor, measuring a change in color formation, light diffraction, light scattering, light adsorption, or light reflection, caused by specific binding of the analyte to the sensor.

13. The device according to claim 1, wherein said sensor uses immunomagnetics to concentrate the analytes on the reaction zone and additionally comprising a means for inducing magnetism in said reaction zone.

14. The device according to claim 1, wherein said sensor uses capillary forces for generating flow of the blood sample through the reaction zone and/or for eliminating non-bound complexes, additionally comprising an absorption pad or a capillary flow inducing means, and optionally a reservoir with fluid, connected to said reaction zone.

15. The device according to claim 7, wherein the electrochemical sensor comprises an enzyme reporting system selected from the group comprising: glucose oxidase, glucose dehydronase, hexokinase, lactate oxidase, cholesterol oxidase, glutamate oxidase, horseradish peroxidase, alcohol oxidase, glutamate pyruvate transaminase, and glutamate oxaloacetate transaminase, horseradish peroxidase/p-aminophenol immunoassay, alkaline phosphatase/1-naphthyl phosphate immunoassay.

16. The device according to claim 7, wherein the electrochemical sensor additionally comprises a combination of an enzyme with an electron transfer mediator.

17. The device according to claim 7, wherein said second sensor is an enzyme-linked immunomagnetic electrochemical assay comprising: an electron-releasing enzyme system coupled to an insulin-specific antibody and secondary insulin-specific antibodies, linked to magnetic particles.

18. The device according to claim 17, wherein upon contact with its substrate, an electron is formed by said enzyme and the current obtained through said enzymatic activity is measured.

19. The device according to claim 17, wherein the magnetic particles are used to capture away the insulin-bound enzyme complexes, and wherein a reduction of electronic current initially present is proportional to the amount of insulin present in the sample.

20. The device according to claim 17, wherein the electron-releasing enzyme system is glucose oxidase.

21. The device according to claim 20, wherein additionally an electron transfer mediator is used such as an ion of ferricyanide.

22. The device according to claim 1, additionally comprising an input means for introducing user-specific data selected from the group comprising: time and/or date of measurement, time of last meal, time and amount of the previous insulin injections, time after exercise, carbohydrate content of the next meal, etc. into said controller, preferably comprising a keypad or a touch-screen.

23. The device according to claim 1, additionally comprising a connection with a computer, portable or mobile processing device, or a smart phone, to enable the user or medical practitioner to follow up his status, insulin need and beta-cell function.

24. The device according to claim 1, which is a home test device or a point of care device.

25. The device according to claim 1, wherein said insulin sensor is specifically detecting long-acting insulin, short-acting insulin, or both, or is specifically detecting C-peptide cleaved from endogenously produced insulin, proinsulin or any form of insulin analogue.

26. The device according to claim 1, wherein the sample receiving part is comprised of a microporous membrane support comprised of a material selected from the group consisting of an organic polymer, inorganic polymer, natural fabrics or synthetic fibers, papers and ceramics.

27.-34. (canceled)

35. A method for calculating the real-time insulin resistance or beta-cell function in a subject, comprising the steps of:
measuring the glucose level in a blood sample of the subject,
measuring the insulin level in a blood sample of the subject, and
calculating the real-time insulin resistance, insulin sensitivity or beta-cell function, based on the measured glucose and insulin levels, using the device according to claim 1.

36. The method according to claim 35, wherein said calculation is done using the HOMA1-IR, HOMA2-IR, Gutt index, Avignon Index, Stumvoll Index, Matsuda Index, HOMA B %, or the Oral Disposition Index formulas.

37. A method for determining the amount of insulin needed in a type-I diabetes mellitus patient comprising the steps of:
detecting the glucose level in a blood sample of a T1DM patient,
detecting the insulin level in said sample, and
calculating the amount of insulin needed in said patient, based on the real-time insulin sensitivity from the combined insulin/glucose level measured, together with the fasting or pre-meal glucose level in the patient and the quantity of carbohydrates in the next meal, preferably using the device according to claim 1.

38. The method according to claim 37, wherein said calculation is done using:
the patient's insulin to carb ratio, to calculate how much insulin is needed to absorb the carbohydrates from the next meal,
the patient's glucose correction factor to calculate how much insulin is needed to correct the fasting or pre-meal glucose level,
both values being corrected for the patient's real-time insulin resistance.

39. A method for determining the amount of Insulin On Board (IOB) in a diabetes mellitus patient comprising the steps of:
detecting the glucose level in a blood sample of a patient,
detecting the insulin level in said sample, and
calculating the amount of Insulin On Board at a given moment based on the previous injected amount of insulin, the time of previous insulin injections and the measured insulin concentration at the time of determining IOB

40. A method for determining the amount of insulin needed in a type-I diabetes mellitus patient comprising the steps of:
detecting the glucose level in a blood sample of a T1DM patient,
detecting the insulin level in said sample, and
calculating the amount of insulin needed in said patient, based on the real-time insulin sensitivity from the combined insulin/glucose level measured, together with the fasting or pre-meal glucose level in the patient and the quantity of carbohydrates in the next meal, subtracting the Insulin On Board estimated by the time delay or more correctly calculated as described under claim 39, preferably using the device according to claim 1.

41. A method for diagnosing or determining the disease state of a type-2 diabetes mellitus patient, an obese subject, a subject with prediabetes or a subject with metabolic syndrome comprising the steps of:
measuring the glucose level in a blood sample of the subject,
measuring the insulin level in a blood sample of the subject, and
calculating the insulin-resistance or beta-cell function based on the level of blood glucose and insulin measured, preferably using the device according to claim 1,
determining the status of the subject, based on said insulin-resistance or beta-cell function, using the device according to claim 1.

42. A method for screening a population of subjects for the being pre-diabetic or for the risk of becoming a diabetic subject, comprising the steps of:
measuring the glucose level in a blood sample of the subject,
measuring the insulin level in a blood sample of the subject, and
calculating the real-time insulin resistance or beta cell function, using the device according to claim 1.

43. The method according to claim 41, wherein said calculation is done using the HOMA1-IR, HOMA2-IR, Gutt index, Avignon Index, Stumvoll Index, Matsuda Index, HOMA B %, or the Oral Disposition Index.

44. A method for better serving the actual basal insulin need of a subject, comprising the step of measuring a real-time insulin sensitivity adapted basal rate of insulin, using a device according to claim 1.

45. A method for better dosing the insulin administration in insulin pump users, comprising the step of measuring real-time insulin sensitivity, using a device according to claim 1.

46. A method for diagnosing subjects and monitoring subjects with overweight, prediabetes or metabolic syndrome comprising the calculation of the beta-cell function in said subjects calculated from the blood glucose and blood insulin levels, determined using a device according to claim 1.
